(19) United States
(12) Patent Application Publication
(10) Pub. No.: US 2009/0035345 A1
(43) Pub. Date: Feb. 5, 2009

(54) Nuclear Receptors Agonists for Treatment of Atherosclerosis and/or Related Cardiovascular Disease

(75) Inventors: Caroline Jacoba Maria De Vries, Amsterdam (NL); Hans Pannekoek, Aalsmeer (NL); Vivian De Waard, Amsterdam (NL); Elisabeth Karin Arkenbout, Ouderkerk aan de Amstel (NL)

Correspondence Address: THE WEBB LAW FIRM, P.C., 700 KOPPERS BUILDING, 436 SEVENTH AVENUE, PITTSBURGH, PA, 15219, US

(73) Assignee: ACADEMISCH MEDISCH CENTRUM, BUREAU, Amsterdam (NL)

(21) Appl. No.: 11/921,968

(22) PCT Filed: Jun. 15, 2006
(86) PCT No.: PCT/EP2006/005764

§ 371 Date: Feb. 27, 2008

(30) Foreign Application Priority Data
Jun. 15, 2005 (EP) PCT/EP2005/006515

(51) Int. Cl. A61F 2/04; A61K 31/404

(52) U.S. Cl. 424/423; 514/414

(57) ABSTRACT

The invention relates to the use of an agonist of one or more of the nuclear receptors TR3, MINOR and NOT for the preparation of a medicament for the treatment of cardiovascular disease, in particular in-stent restenosis and/or vein-graft disease. The invention further relates to medical devices, such as stents and cuffs, that are coated with the agonist, or in which the agonist is incorporated and which are for use in the treatment of in-stent restenosis or vein-graft disease.

The present invention relates to the new use of compounds in the treatment of atherosclerosis and/or atherosclerosis-related cardiovascular disease. The invention in particular relates to the use of said compounds in the treatment of atherosclerosis and/or atherosclerosis-related cardiovascular diseases and/or disorders that involve an excessive proliferation of smooth muscle cells (SMCs), such as in-stent restenosis, vein-graft disease, transplantation arteriosclerosis and arteriovenous shunt failure.

The current widespread use of stents to treat coronary stenosis dramatically increased the incidence of in-stent restenotic lesions. In a sub-population of patients, stent-induced arterial injury is associated with cellular activation and re-entry of smooth muscle cells into the cell cycle, which leads to exuberant cell proliferation and matrix production, and hence luminal narrowing.

Strategies shown to be successful in reducing the rate of in-stent restenosis development, aim at inhibition of smooth muscle cell proliferation. Notably, coating of stents with rapamycin (sirolimus) results in arrest of the cell cycle at the G1/S transition, while coating with paclitaxel induces a mitotic block through stabilization of microtubules. While these approaches present some promise, they also suffer certain limitations, such as the a-specificity of rapamycin and taxanes that inhibit the growth of all cells, including endothelial cells, which prevents proper re-endothelialization of the stented vessel wall, thereby risking undesirable systemic toxic effects and inappropriate healing of the injury.

Bypass surgery is an established intervention to treat coronary artery disease. Coronary artery bypass graft (CABG) surgery restores blood flow to heart tissue that has been deprived of blood because of coronary artery disease. During bypass surgery, a new graft vessel that will carry oxygenated blood around the blockage in a coronary artery is surgically removed from another location in the body. The graft vessel is a healthy artery or vein taken from for example the leg, arm or chest. It is then transferred to the outside of the heart.

Both the saphenous vein and the internal mammary artery are often applied as bypass material. The arterial bypass has a better patency than the venous bypass in which vein-graft disease may develop, resulting in vein-graft failure in 10-30% of the patients per year. Vein-graft disease is the result of excessive smooth muscle cell proliferation that may be caused by mechanical strain. The mammary artery is relatively short, limiting the amount of available bypass material. Therefore, it is vital to improve the function of venous bypasses in terms of enhancement of longevity.

Transplantation arteriosclerosis is the cause of long-term organ failure after organ transplantation and involves excessive smooth muscle cell proliferation in the arteries of the transplanted organ, resulting in concentric intimal lesions that obstruct blood flow.

Arteriovenous shunts are applied in hemodialysis patients and failure of the shunt is caused by disproportionate mechanical stretch of the venous vessel wall, causing vascular smooth muscle cell hyperplasia in the venous compartment of the shunt. Failing arteriovenous shunts are treated with intravascular stent placement.

Smooth muscle cells thus play a key role in vascular pathologies such as the above described (in-stent) restenosis after angioplasty, transplantation arteriosclerosis, vein-graft disease following coronary artery bypass surgery, arteriovenous shunt failure, as well as in atherosclerosis. Even though the first two and last two types of vascular disease occur in the arterial vessel wall and the in the venous vessel wall, respectively, smooth muscle cell hyperplasia is a critical factor in the onset and progression of these large vessel diseases. Various stimuli are involved in initiation of smooth muscle cell proliferation, of which inflammatory pathways involving activated macrophages are well established.

In the research that led to the invention, by genome-wide expression analysis, the inventors revealed that TR3 nuclear orphan receptor (TR3, also named Nur77, NAK1, NGFI-B, NR4A1), and Mitogen-induced nuclear orphan receptor (MINOR, also named NOR-1, NR4A3) and Nuclear orphan receptor of T-cells (NOT, also named nurr1, NR4A2) are among the genes that are specifically induced in macrophages and smooth muscle cells under atherosclerotic conditions. These three genes form a separate subfamily of the nuclear receptor superfamily of transcription factors, which comprises approximately 60 others, including the estrogen receptor and the PPARs. It was shown that TR3, MINOR and NOT are expressed in human atherosclerotic lesions, vein-graft disease, and in in-stent restenosis (ISR) and in porcine arteriovenous shunt-lesions.

To evaluate the function of TR3-like factors during lesion formation in vivo, transgenic mice were generated that express either full-length TR3 or an inhibitor of all three transcription factors (named TR3dTA or ΔTA) under control of a promoter, which directs expression of transgenes to arterial smooth muscle cells. The mice were challenged by a carotid artery ligation, which results in the formation of a smooth muscle cell-rich lesion. Such vascular lesions develop relatively fast and may be considered as the murine model of restenosis. Transgenic mice that express TR3dTA develop a substantial larger neointima compared to wild-type mice. In line with these data, intimal hyperplasia is strongly inhibited in transgenic mice expressing TR3 in arterial smooth muscle cells.

These results unambiguously demonstrate that TR3, and conceivably also MINOR and NOT, inhibit smooth muscle cell-rich lesion formation. Thus, according to the invention it was found that TR3 is a protective factor, which inhibits excessive smooth muscle cell proliferation during vascular lesion formation.

In addition to the expression of TR3, MINOR and NOT in smooth muscle cells, the TR3-like factors (i.e. TR3, MINOR and NOT) are expressed in human atherosclerotic lesions in macrophages and in endothelial cells. The expression of TR3, MINOR and NOT is strongly enhanced upon activation of cultured macrophages, both in primary human macrophages and in the monocytic/macrophage cell line THP-1. Accordingly, it has been demonstrated that TR3-like factors inhibit cytokine and chemokine release of activated macrophages, more specifically the expression of interleukin-1beta, interleukin-6, interleukin-8, monocyte chemotactic protein-1, macrophage inflammatory protein-1alpha and macrophage inflammatory protein-1beta. In addition, in cultured macrophages lipid-loading is inhibited, correlating with reduced expression of scavenger receptor-A and CD36. Consequently TR3-like factors delimit also the formation of complex atherosclerotic lesions. TR3 has also been demonstrated to promote endothelial cell survival and angiogenesis (Zeng H et al., J Exp Med. 2006, 203:719-729), which will facilitate healing processes in the vessel wall after vascular injury, such as for example angioplasty and stent placement.

Based on the above, the stimulation of TR3-like nuclear receptor activity appears to be the key to prevent or reduce atherosclerosis-related cardiovascular disease in general, and in particular the occurrence of in-stent stenosis, vein-graft disease, transplantation arteriosclerosis and arteriovenous shunt failure.

The invention thus relates to the use of an agonist of one or more of the nuclear receptors TR3, MINOR and NOT for the preparation of a medicament for the treatment of atherosclerosis and/or atherosclerosis-related cardiovascular disease.

In particular, the invention relates to the use of an agonist of one or more of the nuclear receptors TR3, MINOR and NOT for the preparation of a medicament for the treatment of in-stent restenosis, vein-graft disease, transplantation arteriosclerosis and/or arteriovenous shunt failure.

According to the invention it has been shown that the transcriptional activity of TR3, MINOR and NOT is regulated via non-traditional agonists such as 1,1-bis(3′-indolyl)-1-(p-substituted phenyl)methanes, containing trifluoromethyl (DIM-C-pPhCF₃), hydrogen (DIM-C-Ph) and/or methoxy (DIM-C-pPhOCH3) substituents, hereafter named “C-DIMs”, which increase the activity of TR3-like factors (Chintharlapalli et al., J Biol. Chem. 2005; 280:24903-24914), which compounds per se, as well as the synthesis thereof, have been described in WO 02/28832.

Accordingly, preferred agonists of the invention are the compounds of the formula:

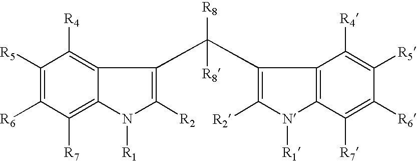

wherein:

R₁, R₂, R₄, R₅, R₆, R₁′, R₂′, R₄′, R₅′, R₆′, and R₇′ are each independently selected from the group consisting of hydrogen, a halogen, a linear C₁-C₁₀ alkyl group, a branched C₁-C₁₀ alkyl group, an alkoxy group containing one to ten carbon atoms, and a nitro group; and

R₈ and R₈′ are each independently selected from the group consisting of hydrogen, a linear C₁-C₁₀ alkyl group, a branched C₁-C₁₀ alkyl group, a cycloalkyl group containing one to ten carbon atoms, and an aryl group.

In a preferred embodiment R₁, R₂, R₄, R₅, R₆, R₁′, R₂′, R₄′, R₅′, R₆′, and R₇′ are each hydrogen, and at least one of R₈ and R₈′ is a branched alkyl group, a cycloalkyl group or an aryl group.

Preferably, R₈ and R₈′ are each individually hydrogen, methyl, C₆H₅, C₆H₄OH, C₆H₄CH₃, C₆H₄CF₃, C₁₀H₇, C₆H₄C₅H₅ or C₆H₄OCH3.

Particular preferred compounds which can be used as TR3, MINOR and/or NOT agonists of the invention are compounds wherein R₁, R₂, R₄, R₅, R₆, R₁′, R₂′, R₄′, R₅′, R₆′, and R₇′ are each hydrogen, and one of R₈ and R₈′ is hydrogen and the other is C₆H₅, C₆H₄CF₃, or C₆H₄OCH3.

In a preferred embodiment, the agonist is a TR3 agonist.

In a specific embodiment of the invention, the treatment is effected by means of a stent that has the agonist incorporated therein and/or coated thereon. A stent is a generally longitudinal tubular device formed of biocompatible material, preferably a metallic or plastic material. A typical stent includes an open flexible configuration. The stent configuration allows the stent to be configured in a radially compressed state for intraluminal catheter insertion into an appropriate site. Once properly positioned within the lumen of a vessel, the stent is radially expanded to support and reinforce the vessel. Radial expansion of the stent may be accomplished by an inflatable balloon attached to the catheter, or the stent may be of the self-expanding type that will radially expand once deployed.

Coatings can be applied by processes such as dipping, spraying, vapour deposition, plasma polymerization, as well as electroplating and electrostatic deposition. The skilled person in the field is very well capable of selecting a coating material that is biocompatible and compatible with the agonist, such as the “C-DIMs”, and such coatings are known in the art. Preferably such coatings have an elution profile that releases the active ingredient over a longer period of time.

Alternatively, the stent itself may be made of a material that has the agonist incorporated therein. This again may be a slow-release material that releases the agonist over a longer period of time.

The stent may also be made of a biodegradable material, which may be coated or may have the agonist incorporated therein. Suitable biodegradable materials that can be used according to the invention are well known to the skilled person.

For the treatment of vein-graft disease the agonist can be applied to the grafted vein in various ways. The agonist can be incorporated in or coated on a cuff that is placed around the vein prior to grafting. Alternatively, the vein may be coated with a liquid that contains the agonist. Suitably such liquid can be solidified to avoid leakage of the agonist away from the vessel. Preferably such solidification can take place prior to implantation of the vein at the site to be treated. An example of a suitable substance is pluronic gel (also known as Pluronic F127 or Poloxamer 407), which is a biocompatible polymer that displays reverse thermal gelation characteristics, that is, the material exists as a liquid at room temperature and as a solid at body temperature. When chilled, pluronic gel is odourless, colourless, and non-greasy. At body temperature it thickens rapidly. The use of pluronic gel to treat vein grafts with aspirin is described by Torsney et al., Circ. Res. 94(11): 1466-1473 (2004).

For the treatment of vein-graft disease the agonist can also be applied by using a polygalactin biodegradable external stent, as described by Vijayan et al., J. Vasc. Surg. 40(5): 1011-1019 (2004)).

The invention thus also relates to a medical device that is capable of eluting an agonist of one or more of the nuclear receptors TR3, MINOR and NOT, in particular the agonists described above, for use in the treatment of atherosclerosis and/or atherosclerosis-related cardiovascular disorders such as in-stent restenosis, arteriovenous shunt failure and/or vein-graft disease. In order to be capable of eluting said agonists, the medical device may be coated with a suitable coating incorporating one or more of said agonists and from which coating the agonists elutes after placement of the device in e.g. the blood vessel. The medical device may also itself comprise a suitable material incorporating one or more of said agonists, from which material the agonists elute.

In a particular preferred embodiment, the medical device is a stent. For the treatment of in-stent restenosis intraluminal stents are preferably used.

In particular for the treatment of vein-graft disease the medical device preferably is a cuff that is capable of eluting an agonist of one or more of the nuclear receptors TR3, MINOR and NOT. According to the invention suitable cuffs are for example made of pluronic gel, incorporating one or more of the agonists of the invention.

The agonists of the present invention can suitably be combined with any other biologically active agent, i.e. drug or other substance that has a therapeutic value, including but not limited to antithrombotics, anticoagulants, antiplatelet agents, thrombolytics, antiproliferatives, anti-inflammatory agents, and other agents that inhibit restenosis, smooth muscle cell inhibitors, antibiotics and the like, and mixtures thereof.

The present invention will be further illustrated in the Examples that follow and that are not intended to limit the invention in any way. In the Examples reference is made to the following figures:

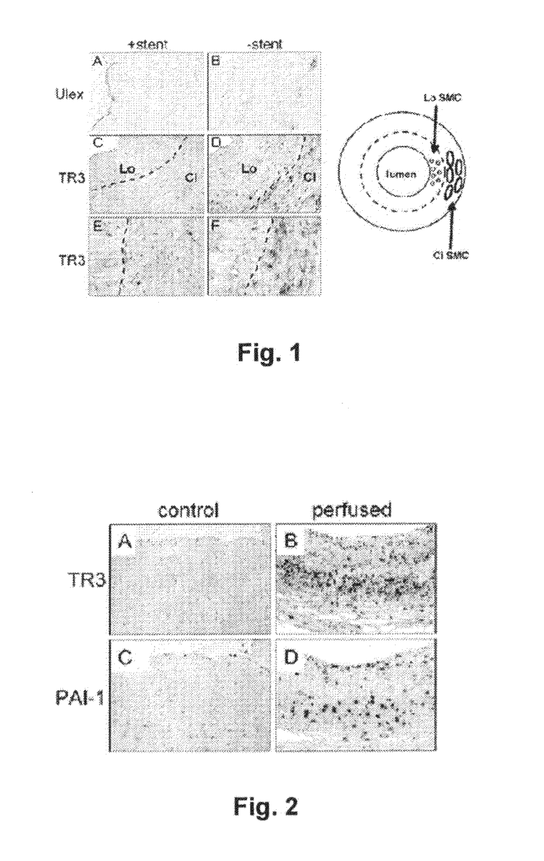

FIG. 1 shows endothelial cell-specific immunohistochemistry and TR3 mRNA expression in perfused vein segments. Vein segments were placed in an extracorporeal bypass loop during bypass surgery and exposed to autologous whole blood flow under arterial pressure for 1 h. Upon perfusion, non-stented vein grafts (B, D, F) displayed overdistension. In vein grafts with an external stent (A, C, E) biomechanical activation was prevented. Vein segments exposed to perfusion at high pressure showed loss of endothelium, whereas capillary endothelial cells (red) were observed near the adventitia as a control for the procedure (B). External stent placement preserved endothelium integrity (A; red monolayer). TR3 mRNA expression was observed by radioactive in situ hybridization (black dots) in the circular (Ci) smooth muscle cell layer in non-stented vein grafts (D 200×; F 400×). Scarce TR3 expression was seen in the stented vein grafts (C 200×; E 400×) or longitudinal (Lo) smooth muscle cell layer (C-F).

The schematic drawing of the venous vessel wall structure shows two distinct smooth muscle cell layers; the Lo and Ci smooth muscle cell layer. The dotted line indicates the border between Lo and Ci smooth muscle cell layer. Nuclei were counterstained in purple (C—F).

FIG. 2 shows TR3 and PAI-1 expression in perfused vein segments. Vein segments were exposed for 6 h to autologous whole blood under arterial pressure (B, D) or instantly fixed to serve as controls (A, C). TR3 mRNA and PAI-1 mRNA expression was detected by radioactive in situ hybridization (black dots) throughout the vein grafts after 6 h of perfusion (B, D), whereas TR3 and PAI-1 expression was only scarcely present in control vein segments (A, C).

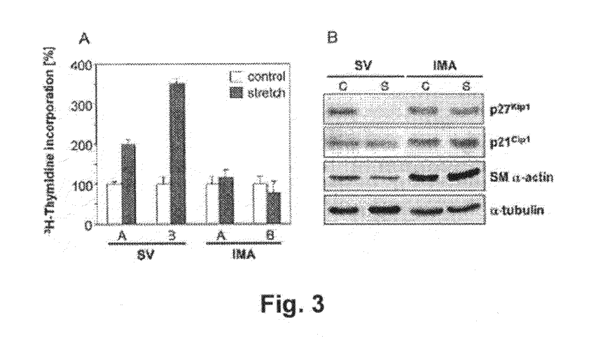

FIG. 3 shows cyclic stretch-induced proliferation in venous smooth muscle cells. DNA synthesis was increased in response to 24 h of cyclic stretch in venous smooth muscle cells derived from two different donors, whereas arterial smooth muscle cells of the same donors were indifferent to stretch (A). [³H]-Thymidine incorporation after stretch was expressed as percentage of control value. p27^{Kip1} was down-regulated after 24 h of stretch in venous smooth muscle cells, while p21^{Cip1} expression levels remained the same as demonstrated by Western Blotting (B). In addition, SM alpha-actin was downregulated in response to stretch in venous smooth muscle cells. In arterial cell lysates the expression of these proteins was unchanged. alpha-Tubulin expression served as control for equal loading. SV indicates saphenous vein smooth muscle cells; IMA, internal mammary artery smooth muscle cells; c, control; s, stretch.

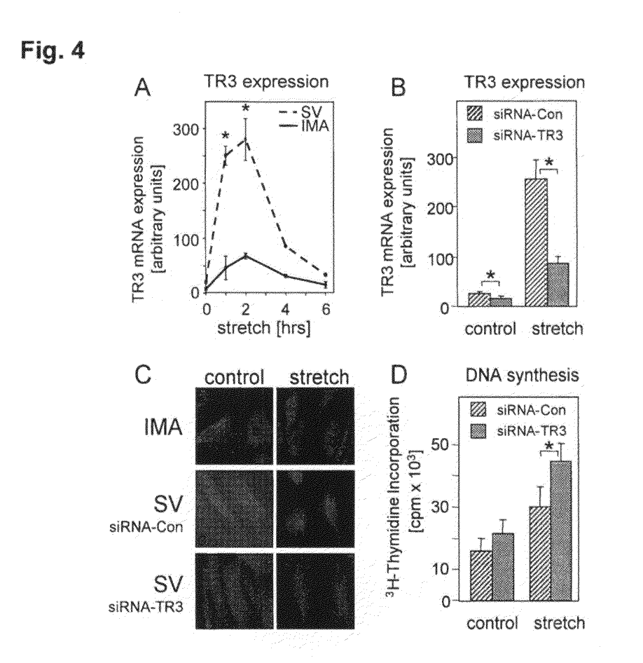

FIG. 4 shows stretch-induced TR3 expression in venous SMCs and enhanced DNA synthesis after TR3 siRNA knockdown. (A) TR3 mRNA expression was increased optimally in venous SMCs 1 to 2 h after stretch. TR3 mRNA expression was corrected for equal mRNA content by correcting for the extent of HPRT mRNA expression. (B) siRNA transfection of venous SMCs resulted in downregulation of TR3 mRNA expression after 2 h of cyclic stretch with TR3 gene-specific siRNA sequences, compared to a control siRNA. (C) TR3 protein expression was detected by immunofluorescence. In IMA SMCs only background signal was detected (a, b). TR3 protein expression is increased in response to stretch (5 h) in SV SMCs (compare c and d) and is downregulated by TR3-siRNA (compare d and f). (D) Knockdown of endogenous TR3 expression by siRNA-TR3 results in enhanced DNA synthesis in response to stretch as measured by [³H]-thymidine incorporation, compared to SMCs transfected with siRNA-Con. SV, saphenous vein SMCs; IMA, internal mammary artery SMCs, *=P<0.01. The results in A were obtained in SV-SMCs derived from 6 independent donors, in B/C the experiments were repeated in 3 distinct SV-SMCs cultures and in D in 5 distinct SV-SMCs cultures.

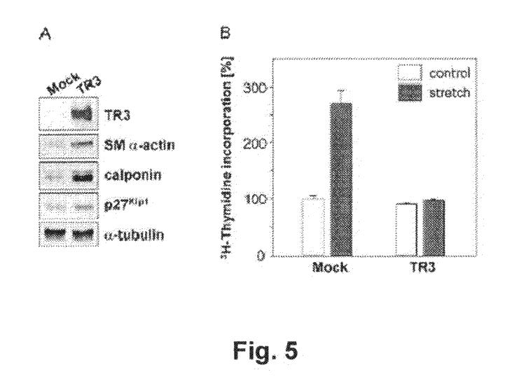

FIG. 5 shows decreased proliferation in venous smooth muscle cells with TR3 adenovirus. The expression of TR3 protein after infection of venous smooth muscle cells with TR3-encoding adenovirus was demonstrated by Western Blotting (A). TR3 was expressed in TR3-infected stretched smooth muscle cells, whereas mock-infected cells did not express measurable endogenous TR3 protein. In the TR3-infected cells SM alpha-actin, calponin and p27^{Kip1} synthesis was more pronounced than in mock-infected cells; alpha-Tubulin served as control for equal loading. [³H]-Thymidine incorporation was increased after 24 h of cyclic stretch in mock virus-infected smooth muscle cells, whereas TR3 virus-infected smooth muscle cells were indifferent to stretch (B). [³H]-Thymidine incorporation was expressed as percentage of the mock control value.

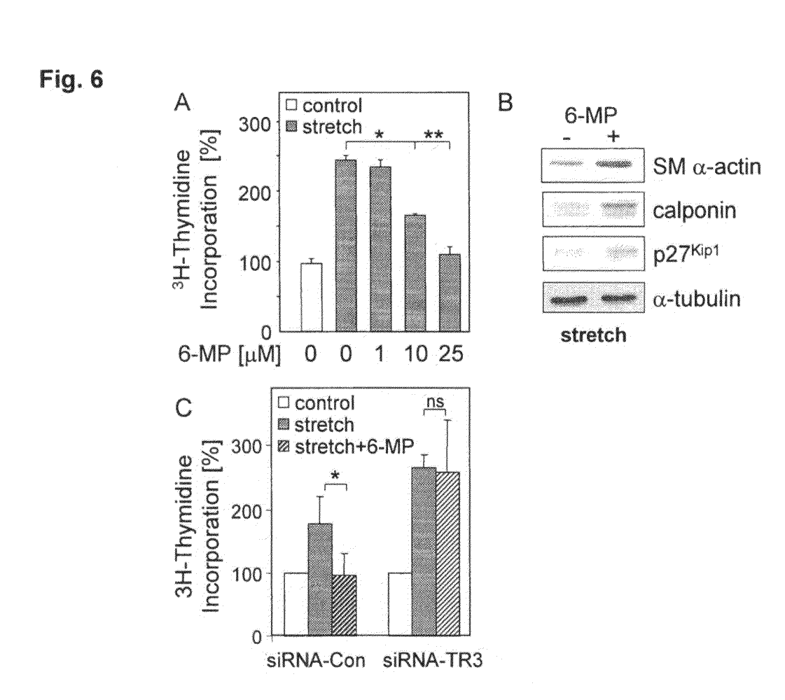

FIG. 6 shows that a TR3-agonist inhibits DNA synthesis in venous SMCs exposed to cyclic stretch.

(A) [³H]-Thymidine incorporation was increased in SMCs in response to 24 h of cyclic stretch, whereas 6-MP reduced stretch-mediated proliferation in a dose-dependent manner. [³H]-Thymidine incorporation was expressed as percentage of the control value. (B) The expression of SMα-actin, calponin and p27^{Kip1} protein in stretched, venous SMCs treated without (−) or with (+) 25 μM 6-MP is detected by Western Blotting. α-Tubulin served as control for equal loading. (C) When TR3 expression is knocked down by siRNA-TR, 6-MP no longer inhibits DNA synthesis, which was measured by [³H]-thymidine incorporation. ANOVA analysis of the data revealed significance of the data. *=P<0.05, **=P<0.01, NS=not significant.

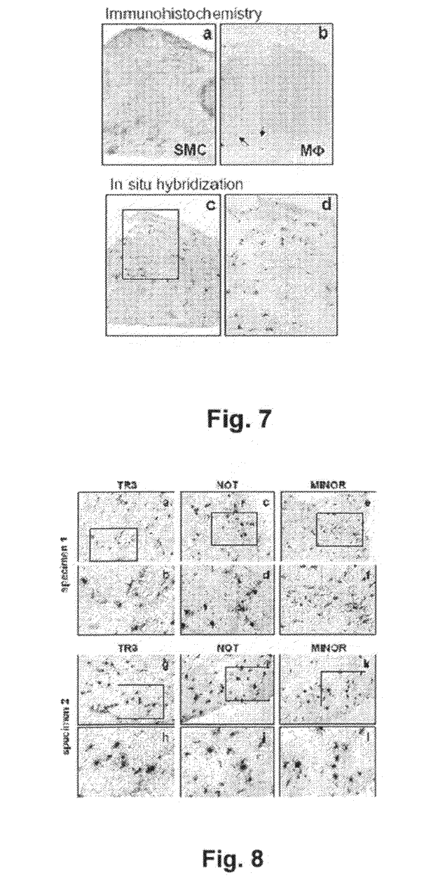

FIG. 7 shows the immunohistochemical analysis of an in-stent restenotic lesion and TR3 mRNA expression. Consecutive sections of an in-stent restenosis specimen were assayed for (a) smooth muscle cell content and (b) the presence of macrophages. Only limited numbers of macrophages were shown to be present. (c, enlargement in d) Radioactive in situ hybridization with a riboprobe specific for TR3, revealed expression throughout the lesion, corresponding with predominant expression in lesion smooth muscle cells. Cells expressing TR3 mRNA contain black spots. Nuclei were counterstained in purple.

FIG. 8 shows the radioactive in situ hybridization to demonstrate TR3, MINOR and NOT mRNA expression in in-stent restenotic atherectomy specimens (specimen 1; a-f and specimen 2; g-l). Scattered expression throughout the lesions was observed for TR3 (a, enlargement in b; g enlargement in h), NOT (c, enlargement in d; i enlargement in j) and MINOR (e, enlargement in f; k enlargement in l). Corresponding sense riboprobes did not show any background (data not shown).

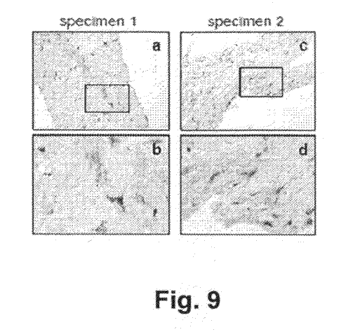

FIG. 9 shows immunohistochemistry to demonstrate TR3 protein expression. Consecutive sections of the specimens shown in FIG. 7 were incubated with an antibody directed against TR3 to reveal a similar pattern of TR3 protein expression (red-brown) in in-stent restenosis as TR3 mRNA; (a, enlargement in b) specimen 1 and (c enlargement in d) for specimen 2.

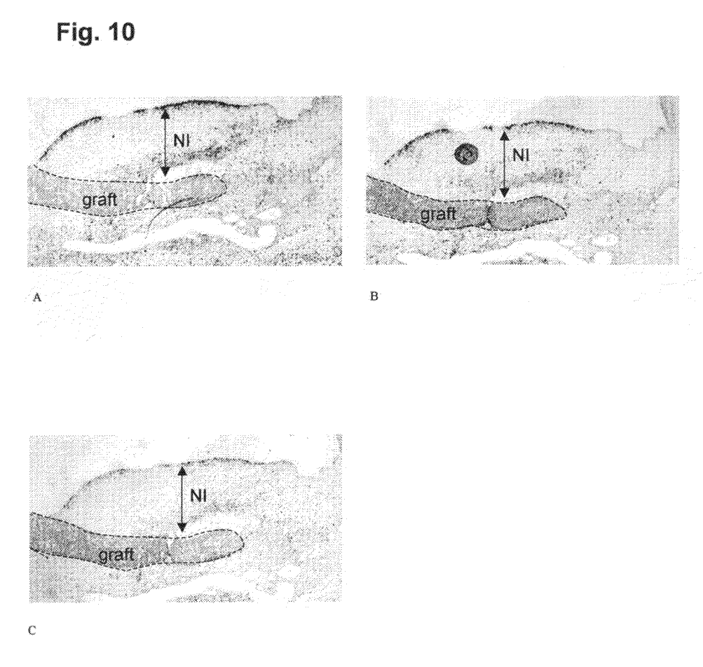

FIG. 10 shows the mRNA expression of TR3, MINOR and NOT by radio-active in-situ hybridization in the neointima and adventitia of pigs, which received an arteriovenous graft for 4 weeks. Corresponding sense riboprobes did not show any background (data not shown). A. TR3 mRNA expression in shoulder region and graft area by radio-active in-situ hybridization (black spots), counterstained with hematoxylin; B. MINOR mRNA expression in shoulder region and graft area by radio-active in-situ hybridization (black spots), counterstained with hematoxylin; C. NOT mRNA expression in shoulder region and graft area by radio-active in-situ hybridization (black spots), counterstained with hematoxylin.

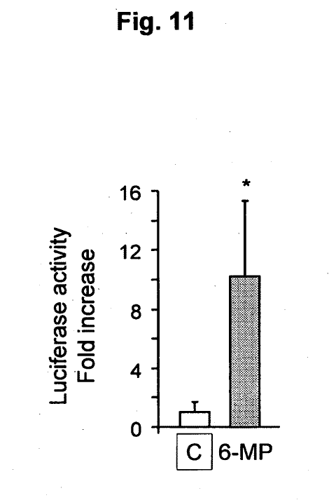

FIG. 11 shows that the Nur77-agonist 6-MP enhances Nur77 activity in cultured SMCs. The transcriptional activity of Nur77 was monitored by measuring luciferase activity in Nur77-expressing SMCs transfected with a Nur77 reporter-luciferase construct, containing the POMC-derived NurRE. Cells were cultured in the absence (C, white bar) or for 24 hours in the presence of 6-MP (6-MP, grey bar) (Mean±SD); *P<0.05.

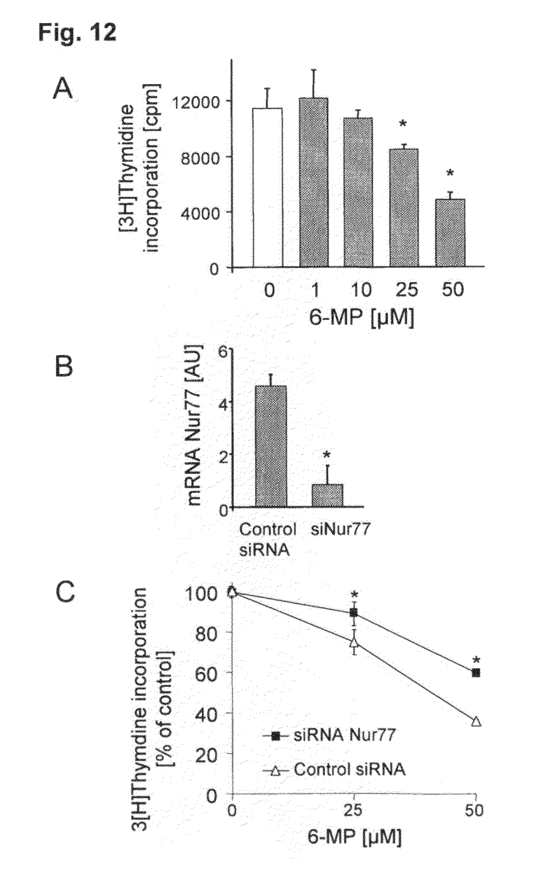

FIG. 12: NR4A agonist inhibits proliferation of cultured SMCs: involvement of Nur77.

A: DNA synthesis of SMCs, grown in medium with vehicle (control, white bar) or indicated concentrations 6-MP (grey bars). DNA synthesis was assayed by [³H]Thymidine incorporation. At 25 μM or 50 μM 6-MP, DNA synthesis is reduced. (Mean±SD, n=3); *P<0.05. B: Nur77 mRNA expression is reduced in SMCs transfected with siNur77 in comparison to SMCs transfected with control siRNA. mRNA levels were determined by real-time RT-PCR and cDNA content of the samples was corrected for P0 expression. (Mean±SD, n=2); *P<0.05 C: The effect of 6-MP on DNA synthesis was determined by [³H]thymidine incorporation in SMCs transfected with control siRNA (white triangles) or transfected with siNur77 (black squares). 6-MP inhibits DNA synthesis less effective in siNur77 transfected cells than in control siRNA transfected cells, demonstrating that the inhibitory effect of 6-MP is at least partly mediated through activation of Nur77 (Mean±SD, n=2); *P<0.05.

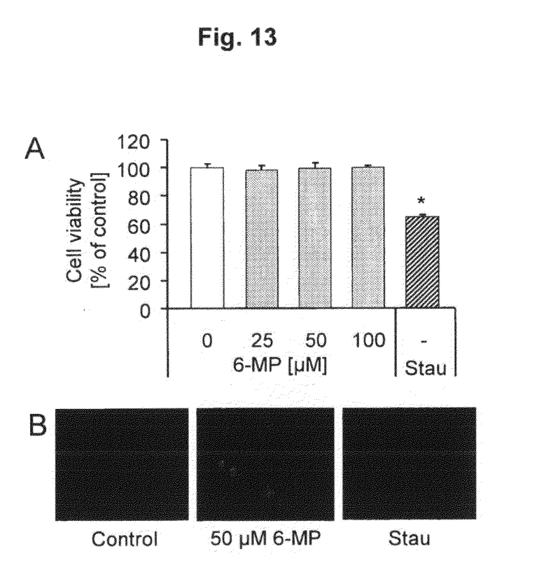

FIG. 13 shows that the agonist 6-MP is not cytotoxic to SMCs and does not induce apoptosis.

A: Viability of SMCs incubated for 24 hours with vehicle (control, white bar), 6-MP (grey bars) or staurosporine (Stau, hatched bar). Viability of cells was determined by MTT assay and expressed as a percentage of control. (Mean±SD, n=3); *P<0.05. B: SMCs were incubated for 24 hours with vehicle, 6-MP or staurosporine. Nuclei were subsequently stained using Hoechst dye. Only staurosporine induces apoptosis and reduces cell viability.

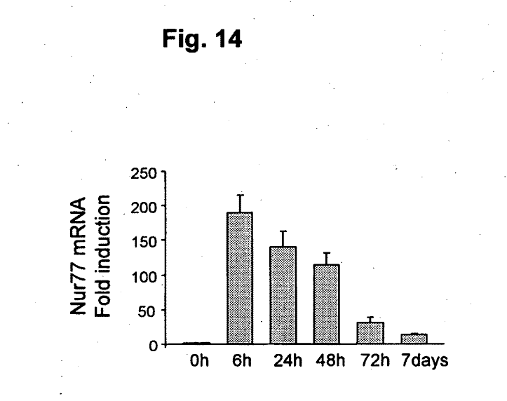

FIG. 14 shows mRNA expression of Nur77 in the murine vessel wall after cuff injury. At different time points (6 hrs up to 7 days) after cuff placement, cuffed vessel segments were harvested and assayed for Nur77 mRNA content. The mRNA expression levels are indicated as the relative expression in comparison to sham-operated vessels. (mean±SEM, n=6). Nur77 mRNA is elevated already 6 hours after injury and remains elevated up to 7 days.

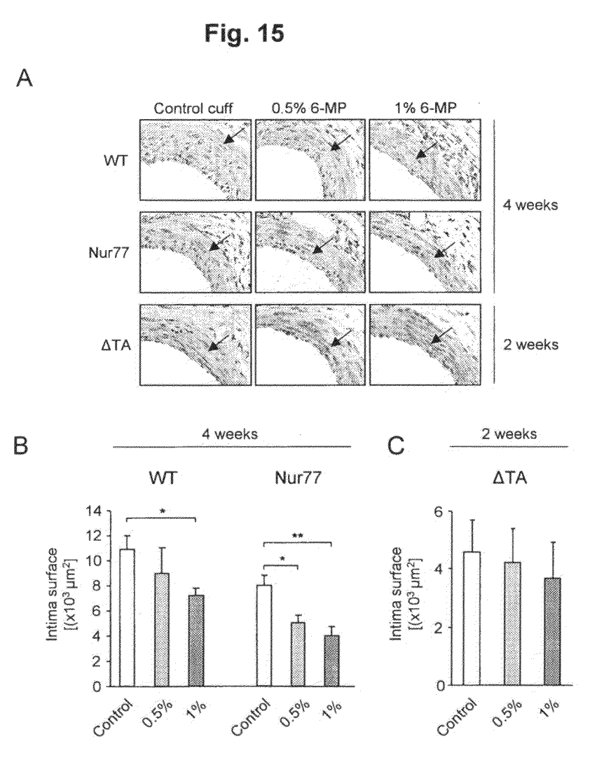

FIG. 15 shows the effect of local NR4A-agonist delivery on neointima formation. A: Representative cross-sections of femoral arteries of wild-type mice (Wt), transgenic mice expressing full-length Nur77 cDNA (Nur77) or mice expressing a dominant-negative variant of Nur77 (ATA), with cuffs containing different amounts of 6-MP. The cuffed vessel segments of Wt and Nur77 transgenic mice were analyzed by HPS staining after 4 weeks and of ATA transgenic mice after 2 weeks (magnification 400×; arrows indicate the internal elastic lamina). B: Morphometric analyses of cuffed vessel segments revealed total intimal area in Wt and Nur77 transgenic mice 4 weeks after placement of cuffs. Cuffs contained either no (control), 0.5% or 1% 6-MP. C: Total intimal area in cuffed femoral arteries in ΔTA transgenic mice 2 weeks after placement of cuffs containing either no, 0.5% or 1% 6-MP. (mean±SEM, n=6);*P<0.05; **P<0.01.

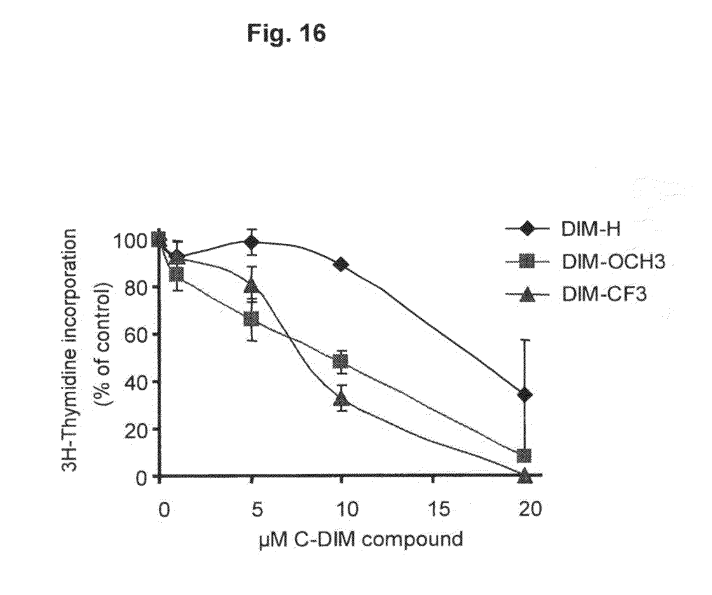

FIG. 16 shows that C-DIM derivatives inhibit proliferation of cultured, human SMCs. DNA synthesis of SMCs was measured by [³H]Thymidine incorporation in the presence of serum and increasing concentrations of C-DIM-H, C-DIM-OCH3 or C-DIM-CF₃. All three C-DIMs inhibit DNA synthesis in SMCs dose dependently. C-DIM-His less effective than C-DIM-OCH3 and C-DIM-CF₃.

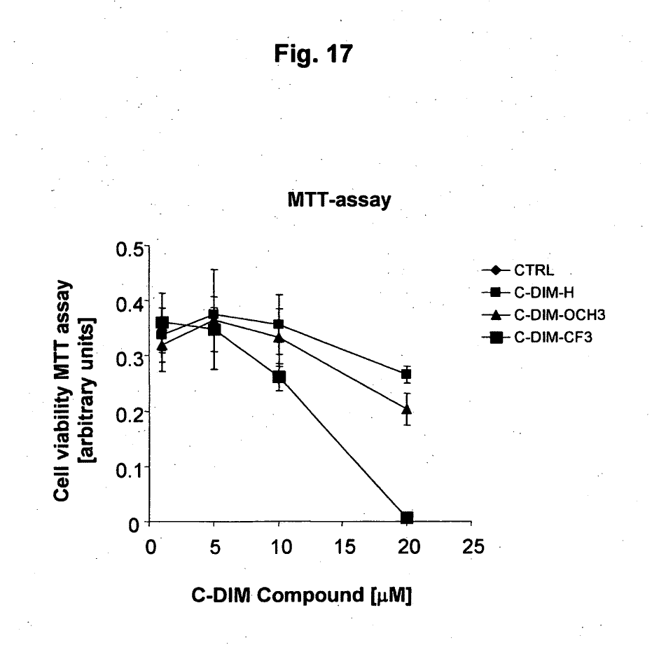

FIG. 17 shows SMC viability, as assessed by MTT assay, at increasing concentrations of C-DIM-derivatives. C-DIM-H and C-DIM-OCH3 do not affect SMC viability up to a concentration of 10 micromolar, whereas C-DIM-CF₃ decreases SMC viability at 10 micromolar and is toxic to the cells at 20 micromolar.

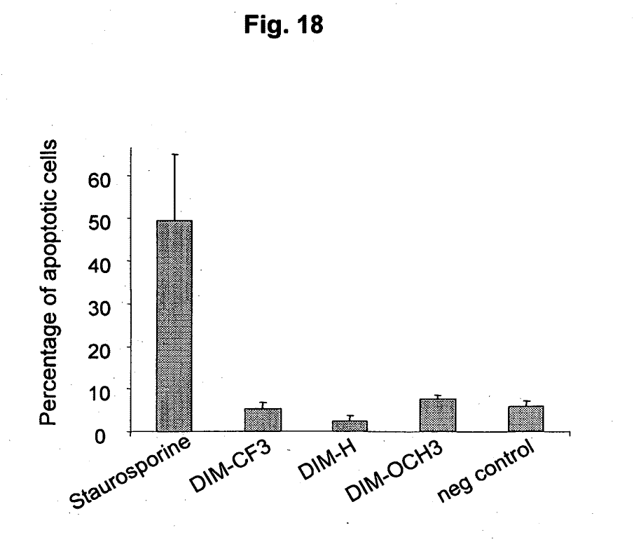

FIG. 18 shows that C-DIM-H, C-DIM-OCH3 or C-DIM-CF₃ do not induce apoptosis in SMCs. SMCs were incubated for 24 hours with vehicle, C-DIM compound (at 10 micromolar) or staurosporine. Nuclei were subsequently stained using Hoechst dye and the percentage of apoptotic nuclei was determined. Only staurosporine induces apoptosis.

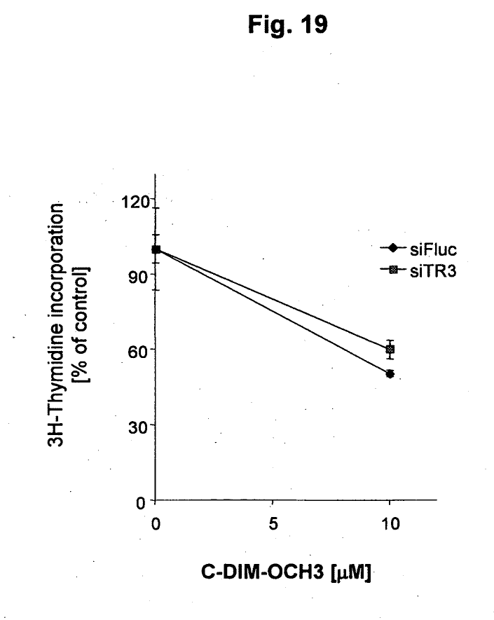

FIG. 19 shows that C-DIM-OCH3 inhibits proliferation of SMCs and the involvement of TR3. DNA synthesis of SMCs was measured by [³H]Thymidine incorporation in the presence of serum. TR3 expression was knocked down by TR3-specific siRNA and it is shown that C-DIM-OCH3 is less effective when TR3 expression is inhibited, demonstrating that the inhibitory effect of C-DIM-OCH3 is at least partly mediated through activation of TR3.

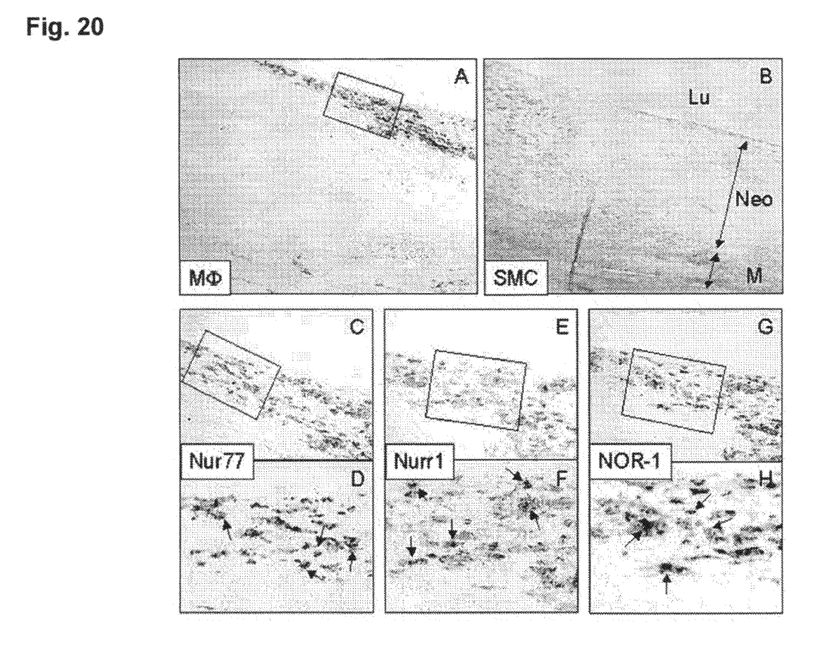

FIG. 20 shows the macrophage-specific expression of Nur77, Nurr1 and NOR-1 in human atherosclerosis. Serial sections of a human type II-lesion (donor III (‡) in Table 1), were analyzed by immunohistochemistry to detect macrophages (A) and SMCs (B). To demonstrate macrophage-specific expression of Nur77, Nurr1 and NOR-1, sections were analyzed simultaneously by macrophage-specific immunohistochemistry and in-situ hybridization with gene-specific probes (C—H). mRNA expression (black silver grains) co-localizes with a number of macrophages (in red) as shown by increased magnification for Nur77 (D), Nurr1 (F) and NOR-1 (H). D, F, H are enlargements of the indicated areas in C, E, G respectively. MΦ, macrophages; Neo, neointima; Lu, lumen; M, media. Arrows in D, F and H point at macrophages expressing the specific mRNAs.

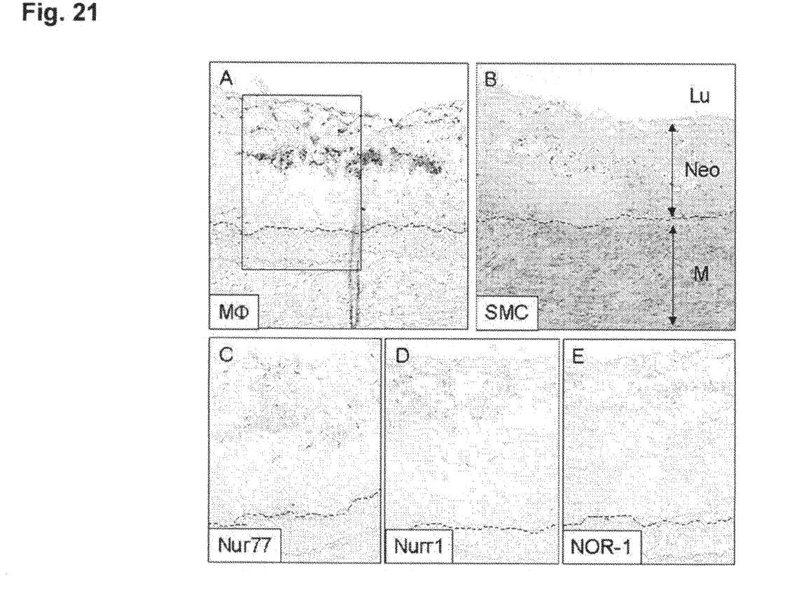

FIG. 21 demonstrates protein expression of Nur77, Nurr1 and NOR-1 in human atherosclerosis. Serial sections of a human type II lesion (donor V (t) in Table 1), were analyzed by immunohistochemistry to detect macrophages (A), SMCs (B), Nur77 (C), Nurr1 (D) or NOR-1 (E). Nur77, Nurr1 and NOR-1 protein is expressed predominantly in neointimal cells and is localized to nuclei. The sections shown in C-E were not counterstained for nuclei. MO, macrophages; Neo, neointima; Lu, lumen; M, media. The dotted lines indicate the internal elastic lamina.

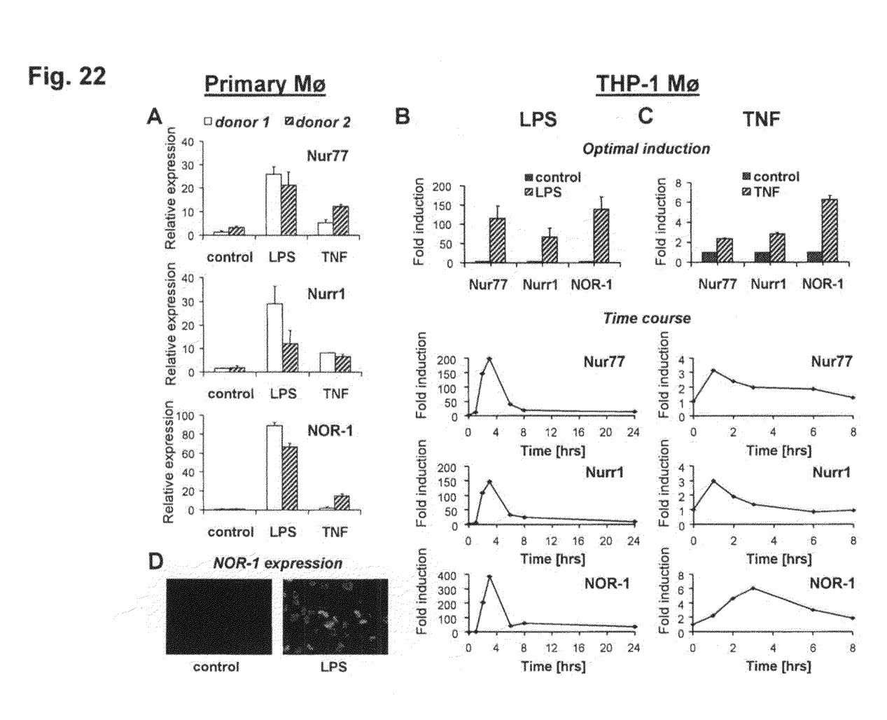

FIG. 22 shows the expression of Nur77, Nurr1 and NOR-1 in primary macrophages and THP-1-derived macrophages in response to LPS and TNFα. mRNA expression levels were determined by real-time RT-PCR. In primary macrophages of 2 different donors treated with LPS (100 ng/ml), TNFα (10 ng/ml) or control for 2 hours increased mRNA expression levels of Nur77, Nurr1 and NOR-1 were observed (A). In THP-1-derived macrophages mRNA expression levels of Nur77, Nurr1 and NOR-1 in response to LPS (250 ng/ml, 2 hours) (B) and TNFα (10 ng/ml, 1 hour for Nur77 and Nurr1, 3 hours for NOR-1) (C) were significantly increased. Optimal expression is shown in the upper panels and time courses are given in the lower panels. Optimal expression experiments (n=3, ±SD, Student's t-test, p<0.05). In time course experiments a representative experiment is shown (n=2). Protein expression of NOR-1 was analyzed in LPS-treated (6 hours) THP-1-derived macrophages by immunofluorescence and localized to the nucleus (D).

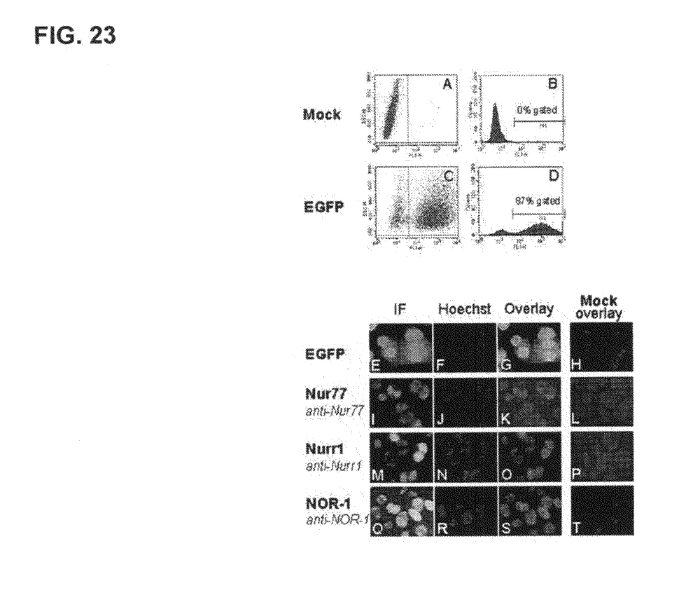

FIG. 23 shows the transduction efficiency of lentiviral infection of THP-1 cells and nuclear localization of the encoded nuclear receptors. THP-1 cells infected with empty lentivirus Mock (A, B) or EGFP-encoding lentivirus (C, D) were analyzed by flow cytometry (A-D). Lentiviral infection resulted in 80-90% transduction efficiency. Simultaneously THP-1 cells infected with recombinant lentivirus encoding EGFP (E-G), Nur77 (1-K), Nurr1 (M-O), NOR-1 (Q-S), or with Mock-virus (H, L, P, and T) were differentiated to macrophages and analyzed for direct fluorescence (EGFP and Hoechst) or immunofluorescence EGFP expression localized throughout the cell, whereas nuclear receptors are predominantly present in nuclei. IF, (immuno)fluorescence.

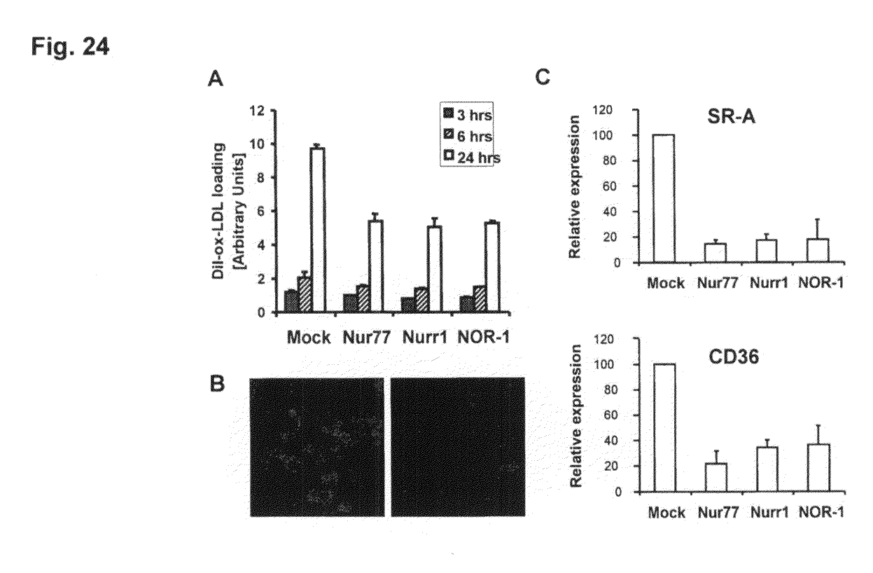

FIG. 24 shows NR4A-factor overexpression in THP-1 derived macrophages reduces DiI-labeled ox-LDL uptake and expression of SR-A and CD36. (A) Uptake of DiI-labeled ox-LDL for 3, 6 and 24 hours was determined by fluorometry. Lipid loading was significantly lower in THP-1 macrophages overexpressing Nur77, Nurr1 or NOR-1 as compared to Mock (n=3, ±SD, Students t-test, p<0.01). (B) After 24 hours of DiI-labeled ox-LDL treatment THP-1 macrophages were analyzed by confocal microscopy showing reduced DiI-fluorescence intensity in Nur77-overexpressing macrophages, localizing to lipid vacuoles. (C) mRNA expression of SR-A and CD36 was determined by real-time RT-PCR. THP-1 macrophages overexpressing Nur77, Nurr1 and NOR-1 expressed significantly lower levels of SR-A and CD36. (B, n=3, ±SD, Student's t-test; p<0.01).

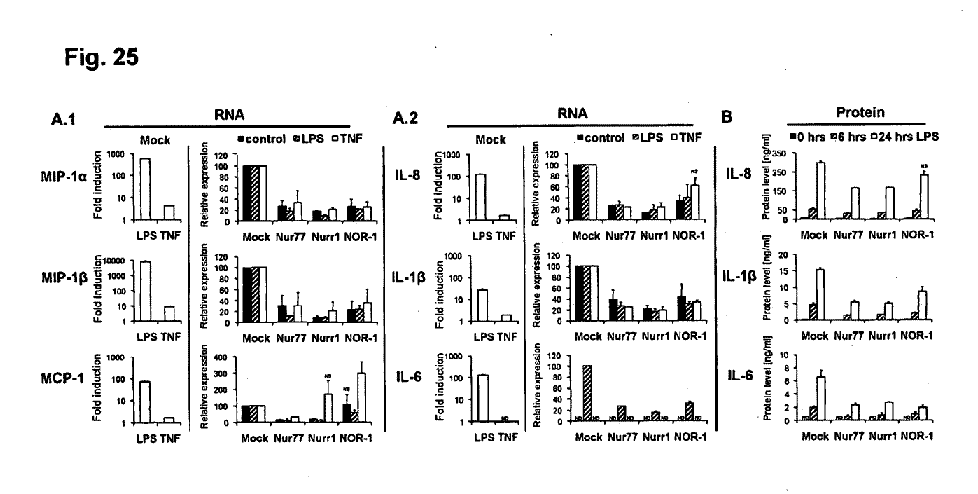

FIG. 25 shows that Nur77, Nurr1 or NOR-1 overexpression reduces inflammatory cytokine and chemokine production. THP-1 macrophages overexpressing Nur77, Nurr1 and NOR-1 were stimulated with LPS (100 ng/ml), TNFα (20 ng/ml) or control for 3 hours and mRNA levels of MIP-1α, MIP-1β and MCP-1 (A.1) and IL-1, IL-6 and IL-8 (A.2) were determined by real-time RT-PCR. In Mock-lentivirus infected cells all genes analyzed were induced 20-8000 fold after LPS and 3-10 fold after TNFα (except for IL-6, not detectable after TNFα (ND)). THP-1 macrophages overexpressing Nur77, Nurr1 and NOR-1 expressed significantly lower mRNA levels (>50% reduction) of most of the genes analyzed (n=3, ±SD, Student's t-test, p<0.05). Protein levels of IL-8, IL-1β and IL-6 were determined in conditioned media (B). Supernatant was collected at 0, 6 and 24 hours after treatment with LPS. Protein levels of IL8, IL-1 and IL-6 were significantly reduced in THP-1 macrophages overexpressing Nur77, Nurr1 and NOR-1s (n=3, ±SD, Student's t-test, p<0.05; ND: not detectable; NS: not significant).

### EXAMPLES

### Example 1

### TR3 Nuclear Orphan Receptor Prevents Cyclic Stretch-Induced Proliferation of Venous Smooth Muscle Cells

To define the relative contribution of cyclic stretch in initiation of vein-graft disease and to delineate the underlying mechanism of this stimulus in venous SMC hyperplasia compared to SMCs derived from the internal mammary artery, the expression of the early-response gene TR3 was studied in distinct SMC stretch models.

First, cultured SMCs derived from both saphenous veins and internal mammary arteries were exposed to cyclic stretch and it was shown, in accordance with published data, that venous SMCs become proliferative whereas arterial SMCs remain quiescent.

Second, both human and (transgenic) mouse vessels were studied in dedicated organ culture models in which arterial (pulsatile) pressure was applied.

Third, functional involvement of TR3 in inhibition of stretch-induced proliferation was demonstrated by overexpressing the gene, inhibiting the expression of endogenous TR3 with siRNA and by enhancing TR3 activity with 6-MP, a TR3 agonist.

### Materials and Methods

### Human Tissue Specimens

The ex vivo perfusion model in which human saphenous vein segments were exposed to whole-blood under arterial pressure was used as described previously (Stooker et al., J. Thorac. Cardiovasc. Surg., 121: 290-297, 2001). Briefly, vein segments were placed in a loop of the extracorporeal circulation during bypass surgery and were exposed to autologous blood under flow (non-pulsatile) and arterial pressure (60 mm Hg). To study the effect of overdistension on bypass veins, vein segments were perfused in the presence or absence of an external stent. After one and six hours of perfusion the vein segments were harvested, fixed in formalin and embedded in paraffin for histological examination. Patients included in this study gave their informed consent and the study was approved by the local medical ethical committee. Anaesthesia and cardiopulmonary bypass surgery were performed according to routine protocols.

### In Situ Hybridization

In situ hybridizations were performed as described in Boot R G et al. (Arterioscler Thromb Vasc Biol., 19:687-94, 1999). TR3 and PAI-1 probes were synthesized: TR3, GenBank No. L13740, base pairs (bp) 1221 to 1905; PAI-1, GenBank No. X12701, bp 52 to 1308. The probes were labeled with [³⁵S]-UTP (Amersham Biosciences, Buckinghamshire, U.K.).

Paraffin sections (5 microm) of control and perfused saphenous vein segments were mounted on SuperFrost Plus slides (Menzel-Glaser, Braunschweig, Germany). After hybridization and stringent washes, the in situ sections were covered with nuclear research emulsion (ILFORD Imaging UK Limited, Cheshire, U.K.), exposed for 2 to 9 weeks, then developed and counterstained with hematoxylin and eosin.

Matching sense riboprobes were assayed for each gene and were shown to give neither background nor aspecific signal. As a control for the integrity of RNA, in situ hybridizations were performed with an antisense riboprobe for thrombin receptor PAR-1 (Genbank M62424 bp 3076-3472). PAR-1 was abundantly expressed in smooth muscle cells of control and perfused vein segments, indicating that the integrity of the RNA was comparable in all specimens (data not shown).

### Immunohistochemistry

Paraffin sections (5 microm) were deparaffinized, rehydrated and incubated with 0.3% (v/v) hydrogen peroxide and blocked with 10% (v/v) pre-immune goat serum (DAKO, Glostrup, Denmark) in 10 mM Tris-HCl (pH 8.0), 150 mM NaCl (TBS). Subsequently, sections were incubated overnight at 4° C. with biotinylated Ulex Europaeus Agglutinin (Vector Laboratories, Inc. Burlingame, Calif.) (1:50 dilution) in TBS, followed by detection with streptavidin-horseradish peroxidase conjugates (DAKO) and, subsequently, with amino-ethylcarbazole and hydrogen peroxide. Cultured cells were fixed with methanol and stained for SM alpha-actin with monoclonal antibody 1A4 (1:200; DAKO), and biotinylated goat anti-mouse secondary antibodies (DAKO). After counterstaining with hematoxylin, sections were embedded in glycergel (Sigma, St. Louis, Mo.). Immunofluorescent nuclear staining was performed with Hoechst 33258 (Sigma).

### Smooth Muscle Cell Culture

Venous and arterial smooth muscle cells were cultured from explants of saphenous vein (SV) and internal mammary artery (IMA) in Medium 199 with HEPES containing 20% (v/v) fetal bovine serum (FBS) with penicillin and streptomycin (GIBCO, Invitrogen Life Technology, Breda, The Netherlands) and were used at passages 4 to 6. smooth muscle cells were characterized with monoclonal antibody 1A4, directed against SM alpha-actin (DAKO) and demonstrated homogenous fibrillar staining. Overnight incubation with 10 microM carbonyl cyanide chlorophenylhydrazone (CCCP) induced smooth muscle cell apoptosis.

To study stretch-induced responses, smooth muscle cells were seeded in 6-well plates containing collagen Icoated flexible membranes (BioFlex® culture plates, Dunn Labortechnik GmbH, Asbach, Germany) and were stretched in the Flexercell FX3000 apparatus (Dunn Labortechnik) for 1, 2, 4, 6, or 24 h at 10% stretch at 0.5 Hz or served as control (without stretch). Silicone-based lubricant was applied to prevent friction between the membrane and loading post.

[³H]-Thymidine incorporation, adenovirus infection, siRNA electroporation and 6-MP treatment of SMCs:

Smooth muscle cells were seeded in 6-well stretch plates and when wells were confluent, smooth muscle cells were made quiescent for 16 h in medium containing 0.5% (v/v) FBS.

The plates were transferred into the Loading Station™ and stretched for 24 h. Control plates, without stretch, were cultured under identical conditions. Thereafter, cells were labeled for 4 h with 0.5 microCi/mL [methyl-³H]-thymidine (Amersham Biosciences).

Incorporated radioactivity was precipitated for 30 min at 4° C. with 10% (wt/v) trichloroacetic acid, washed twice with 5% (wt/v) trichloroacetic acid and dissolved in 0.5N NaOH. [³H]-thymidine was measured by liquid scintillation counting.

When cells were infected with mock- or TR3-containing adenovirus (3×10⁸ plaque-forming units) for 2 h, the cells were allowed to recover for 24 h in complete medium before they were made quiescent. Agonist treatment (6-MP) was initiated 1 h prior to stretch with 0, 1, 10, 25 microM C-DIM (stock at 40 mM in DMSO).

The following small interfering RNA (siRNA) sequences were used: TR3 siRNA, 5′-CAG UCC AGC CAU GCU CCU C dTdT-3′, and mutated control siRNA, 5′-CAG ACG AGC CUU GCU CGU C dTdT-3′ (Ambion Inc., UK). Per Flexerplate-well 1 μg of siRNA was transfected into 5×10⁵ SMCs using Nucleofector reagent for SMCs (Amaxa GmbH, Cologne, Germany) as per the manufacturer's recommendations and subsequently the cells were placed in the stretch plates and were treated as described above.

### Western Blotting Analysis

Sodium dodecyl sulfate-polyacrylamide gel electrophoresis was performed with cell lysates (30 microg per lane) and concentrated culture media (equivalent of 200 microl per lane). Proteins were transferred to nitrocellulose-Protran (Schleicher and Schuell, 's-Hertogenbosch, The Netherlands).

Expression of p27^{Kip1} (BD Biosciences, Alphen a/d Rijn, The Netherlands), p21^{Cip1} (BD), SM alpha-actin (DAKO), PAI-1 (MAI-12; Biopool, Umea, Sweden), TR3 (M-210; Santa Cruz Biotechnology, Santa Cruz, Calif.), calponin (clone hCP; Sigma) and alpha-tubulin (Cedar Lane, Hornby, Ontario, Canada) was studied, using the indicated antibodies directed against these proteins. Primary antibodies were incubated overnight at 4° C. in 5% Protifar plus (Nutricia, Cuijk, The Netherlands) in TBS. As secondary antibodies, horseradish peroxidase-conjugated goat anti-rabbit (for p27^{Kip1} and TR3 detection) or goat anti-mouse (for all others) (BioRad 11 Laboratories Inc., Hercules, Calif.) in a dilution of 1:5000 in TBS were used.

Proteins were visualized by enhanced chemiluminescence detection (Lumi-Light^{PLUS}; Roche Diagnostics GmbH, Mannheim, Germany). Quantitative analysis was performed by the Lumi-Imager (Boehringer Mannheim, Mannheim, Germany). alpha-Tubulin staining served as a control for loading.

### Real-Time RT-PCR

Total RNA was isolated using Trizol reagent (GIBCO). cDNA was synthesized by reverse transcription (RT) from 1 microg of total RNA with SuperScript II (GIBCO) and 0.5 microg (dT) 12-18 primer. Real-Time polymerase chain reaction (PCR) was performed with the use of the FastStart DNA Master SYBR green I kit (Roche) in the LightCycler System (Roche). Primers for TR3 were as follows: (forward) 5′-GTTCTCTGGAGGTCATCCGCAAG-3′ and (reverse) 5′-GCAGGGACCTTGAGAAGGCCA-3′. As a control for equal amount of first strand cDNA in different samples we determined hypoxanthine phosphoribosyl transferase (HPRT) mRNA levels with primers

(forward)
5′- TAATTATGGACAGGACTGAACG-3′
and
(reverse)
5′- CACAATCAAGACATTCTTTCCAG-3′.

### Results

### TR3 Expression in Perfused Saphenous Vein Segments

To study the molecular processes causing vein-graft disease, an ex vivo perfusion model was applied in which segments of saphenous veins were placed in the extracorporeal circulation during coronary artery bypass surgery. During perfusion significant distension was observed in the non-stented saphenous veins which resulted in an almost complete loss of the endothelial cell layer after already 1 h of perfusion under arterial pressure.

Veins protected against excessive mechanical strain due to placement of an external stent contained intact endothelium after perfusion (as illustrated by endothelium-specific immunohistochemistry, FIG. 1A).

In the non-stented vein segments endothelial cell-specific staining revealed the presence of endothelial cells in capillaries at the adventitia, whereas the luminal endothelium had disappeared (FIG. 1B).

The structure of saphenous veins differs in smooth muscle cell organization from the arterial wall, as veins contain two smooth muscle cell layers that are oriented in opposite directions. A layer of longitudinally oriented smooth muscle cells is situated close to the lumen of the vessel and a circular smooth muscle cell layer (like in arterial vessels) is present adjacent to the adventitia (FIG. 1, schematic drawing). In search for genes involved in vein-graft disease mRNA expression of early response gene TR3 was assayed in ex vivo perfused vein segments by radioactive in situ hybridization. After 1 h of perfusion, TR3 expression was detected in occasional endothelial cells and smooth muscle cells in the stented vein segments (FIG. 1C, E). However, extensive TR3 expression was detected predominantly in the circular smooth muscle cell layer of the non-stented vein segments (FIG. 1D, F). TR3 expression was virtually absent in the control vein segment (FIG. 2A). Yet, after 6 h of perfusion TR3 was abundantly expressed throughout the entire vessel, in both the longitudinal and circular smooth muscle cell layers, in the non-stented perfused vein (FIG. 2B).

In addition, PAI-1 mRNA expression was analyzed since at present PAI-1 is the only known gene that is both related to vascular biology and has a functional TR3 response element. PAI-1 was present in occasional endothelial cells and smooth muscle cells in control veins (FIG. 2C) and after 1 h of perfusion (data not shown). However, PAI-1 expression was strongly increased in smooth muscle cells after 6 h of perfusion (FIG. 2D).

In conclusion, TR3- and PAI-1 mRNA are expressed in smooth muscle cells in saphenous vein grafts subjected to perfusion under arterial pressure and TR3 mRNA expression is initially localized in the circularly oriented SMCs. The circular SMC layer is the outer part of the venous vessel wall indicating that TR3 expression is presumably not induced by a circulating factor in blood or in response to endothelial cell damage, but rather that the key stimulus is cyclic stretch.

### Cyclic Stretch-Induced Proliferation in Venous Smooth Muscle Cells

To investigate why mammary artery bypass material has a better patency than bypass material derived from saphenous vein, the intrinsic difference between smooth muscle cells derived from these different vessels was studied in response to mechanical strain. For the in vitro stretch experiments an experimental stretch-device (Flexercell FX-3000 apparatus) was applied in which all cells are exposed to the same extent of stretch. Standardization of this stretch model involved analysis of DNA synthesis.

Smooth muscle cells, derived from mammary artery or saphenous vein origin, were subjected to 10% cyclic stretch (0.5 Hz) for 24 h and [³H]-thymidine incorporation was measured. It was observed that stretch induced DNA synthesis in venous smooth muscle cells (2 to 3.5 fold, dependent on donor A or B), whereas arterial smooth muscle cells derived from the same individuals remained quiescent (FIG. 3A).

To further substantiate changes in cell-cycle progression, the expression level of cell-cycle proteins was analyzed in cell lysates of stretched smooth muscle cells of venous and arterial origin. Cyclin-dependent kinase inhibitor p27^{Kip1} was found to be decreased upon stretch in venous smooth muscle cells (FIG. 3B). In contrast, stretch did not alter the expression of p27^{Kip1} in arterial smooth muscle cells. The expression of another cell-cycle inhibitor, p21^{Cip1}, was not affected by stretch in both venous and arterial smooth muscle cells. SM alpha-actin expression was assayed as a marker for quiescent smooth muscle cells and was moderately decreased in venous smooth muscle cells after stretch (FIG. 3B).

In conclusion, cyclic mechanical stretching induced the proliferative phenotype in saphenous vein smooth muscle cells, while mammary artery smooth muscle cells remained quiescent.

### Cyclic Stretch-Induced TR3 Expression in Venous Smooth Muscle Cells

To extend the observations made in SV segments exposed to perfusion, it was determined whether TR3 mRNA is also expressed by in vitro cultured SMCs upon cyclic stretch. Saphenous vein and mammary artery SMCs were stretched for periods of 1, 2, 4 or 6 h, while non-stretched cells served as controls. TR3 mRNA was up-regulated in arterial SMCs (FIG. 4A). However, in venous SMCs, TR3 mRNA expression was induced 14.2+/−1.7 fold after 1 to 2 h cyclic stretch to a significantly higher level than in arterial SMCs. TR3 protein expression was analyzed by immunofluorescence (FIG. 4C, a-d), demonstrating only background signal in mammary artery SMCs without and with stretch (a, b). In saphenous vein SMCs transfected with a control siRNA, TR3 protein expression is robustly induced after 5 h of stretch (c, d). Again, mammary artery-derived SMCs appear to be distinct from venous SMCs and seem less responsive to cyclic stretch.

In previous studies we described and applied a dominant-negative variant of TR3 (ΔTA) that inhibits the activity of all TR3-like factors. In the current study we chose to specifically knockdown TR3 expression in venous SMCs by TR3-specific siRNA. Clearly, TR3-specific siRNA reduced endogenous TR3 mRNA expression after 2 h of cyclic stretch to approximately 30% of the expression in the presence of a control siRNA (FIG. 4B). TR3 protein expression was also reduced by siRNA knock down as shown in FIG. 4C by immunofluorescence (compare d and f). Significantly, this reduction in TR3 expression resulted in an increased proliferative response of the cells in response to stretch as shown by [³H]-thymidine incorporation experiments (FIG. 4D).

### Adenoviral Expression of TR3 Decreased Proliferation in Venous Smooth Muscle Cells

To evaluate functional involvement of TR3 in the response of venous smooth muscle cells to mechanical strain, TR3 was overexpressed applying adenoviral infection. TR3 protein expression in stretched smooth muscle cells, was confirmed by Western blotting analysis (FIG. 5A). Even after stretch, TR3 virus-infected smooth muscle cells showed a more differentiated (contractile) smooth muscle cell phenotype reflected by increased synthesis of SM alpha-actin, calponin and p27^{Kip1} protein when compared to mock virus-infected cells (FIG. 5A).

After 24 h of stretch, the virus-infected cells were assayed for DNA synthesis by [³H]-thymidine incorporation. Mock virus-infected cells showed a similar response as the non-infected venous smooth muscle cells (compare with FIG. 3A) [³H]-thymidine incorporation was induced 2.7-fold upon stretch (FIG. 5B). TR3-infected smooth muscle cells did not proliferate under conditions of cyclic stretch as revealed by an equal amount of [³H]-thymidine incorporation in control and stretched TR3-infected smooth muscle cells.

In conclusion, TR3 overexpression prevents the differentiation to a proliferative phenotype.

Decreased proliferation in venous smooth muscle cells by TR3-Agonist Treatment

To determine whether 6-mercaptopurine (6-MP), a known TR3 agonist, influences stretch-induced proliferation, venous SMCs were treated with 6-MP at various concentrations. Untreated venous SMCs, subjected to 24 h of stretch, showed a 2.5 fold induction of [³H]-thymidine incorporation, whereas the effect on DNA synthesis was reduced in a dose-dependent manner by 6-MP treatment (FIG. 6A). At 25 μM 6-MP, stretch-induced DNA synthesis was completely inhibited. Analogous to TR3 overexpression, 6-MP also increases SMα-actin and calponin as well as p27^{Kip1} protein expression under stretch conditions (FIG. 6B). To reveal the relative contribution of TR3 in 6-MP-mediated inhibition of stretch-induced proliferation, we assayed the effect of 6-MP on DNA synthesis in SMCs transfected with TR3-siRNA (FIG. 6C). Knockdown of TR3 by siRNA completely abolishes the effect of 6-MP on stretch-induced DNA synthesis. These data unambiguously demonstrate that TR3 mediates the inhibitory effect of 6-MP on the proliferative response of SMCs in stretch, and that agonists of TR3, such as the agonists according to the invention, the “c-DIMs”, are very promising drug candidates in the treatment of atherosclerosis-related cardiovascular diseases.

### CONCLUSION

In conclusion, vein-graft disease is the result of excessive SMC proliferation in response to biomechanical stimulation of venous bypass grafts. Venous SMCs respond to cyclic stretch by initiation of proliferation, while at the same time also cell-cycle inhibitory feedback systems are activated, such as the recently described for the IEX-1 pathway (Schulze et al., Circ. Res. 93: 207-212, 2003) and the TR3 transcription factor pathway as identified in this study. The activity of endogenous TR3 is enhanced by 6-MP, which shows that agonists of TR3, such as 6-MP, may modulate biomechanical intimal thickening after bypass surgery as a means to prevent excessive SMC proliferation and subsequent vein-graft disease. The above data thus show that TR3 may act as a target for intervention in vein-graft disease.

### Example 2

### Expression of Nuclear Receptors TR3, MINOR and NOT in In-Stent Restenosis and in Porcine Arteriovenous Shunt Lesions

In the current example, expression of TR3-like factors in in-stent restenosis and in porcine arteriovenous shunt lesions was studied by in situ hybridization and immunohistochemistry.

### Materials and Methods

### Human Tissue Specimens

Human tissue samples were obtained, with informed consent, from patients undergoing directional coronary atherectomy for in-stent restenosis, according to protocols approved by the Medical Ethical Committees of the Academic Medical Center, Amsterdam and the University of Groningen, Groningen (The Netherlands). The retrieved specimens were immediately frozen in liquid nitrogen, stored at −80° C., and 5-mm sections were mounted on Superfrost plus glass slides for immunohistochemistry and in situ hybridization (Emergo, Tournai, Belgium).

### Porcine Tissue Specimens

Female Landrace pigs received arteriovenous grafts (AV graft) bilaterally between the carotid artery and the jugular vein using expanded polytetrafluoroethylene (ePTFE). After 4 weeks the grafts and adjacent vessels were perfused with saline and subsequently with formalin at physiologic pressure. Subsequently, grafts and adjacent vessels were excised and immersed in formalin for at least 24 h after which 5-mm blocks were paraffin embedded. Of the retrieved specimens 5-um sections were mounted on Superfrost plus glass slides for in-situ hybridization (Emergo, Tournai, Belgium). The pig-model and graft neointimal lesion histology are described in detail by Rotmans J L et al., Journal of Surgical Research 113, 161-171 (2003) and Circulation 111, 1537-42 (2005). The model is used as a model for arteriovenous graft failure (in other words stenosis).

### In Situ Hybridization

Radioactive in situ hybridization was performed as described previously. The following riboprobes were used: TR3, Genbank L13740, basepairs (bp) 1221-1905; MINOR, Genbank U12767, bp 1435-2172; NOT, Genbank X75918, bp 119-1003. Matching sense riboprobes were assayed for each gene and were shown to give neither background nor an aspecific signal.

### Immunohistochemistry, Western Blotting and Immunoradiometric Assay

TR3 antigen was detected by immunohistochemistry with a rabbit antiserum, directed against Nur77 (M-210, Santa Cruz Biotechnology, Calif.).

### Results

### TR3, MINOR and NOT Expression in Atherectomy Specimens

Nine in-stent restenosis specimens, obtained by intravascular directional atherectomy in coronary arteries of nine different patients, were examined by in situ hybridization and immunohistochemistry for expression of the nuclear receptors TR3, MINOR and NOT. Table 1 shows the results.

TABLE 1

| | | | percentage of cells expressing | | |
|---|---|---|---|---|---|
| | Gender | age | TR3 | NOT | MINOR |
| 1 | Female | 75 | 38 | 35 | 25 |
| 2 | Male | 68 | 31 | 25 | 16 |
| 3 | Male | 68 | 31 | 23 | 33 |
| 4 | Male | 57 | 13 | 27 | 7 |
| 5 | Male | 75 | 17 | 18 | 18 |
| 6 | Male | 62 | 2 | 48 | nd |
| 7 | Female | 48 | 45 | 28 | 4 |
| 8 | Male | 69 | 78 | 50 | 21 |
| 9 | Female | 48 | 43 | 70 | 13 |

The main cell type present in in-stent restenotic lesions is the smooth muscle cell as is shown in FIG. 7a by smooth muscle cell-specific immunohistochemistry. However, in some areas scattered macrophages are present (FIG. 7b). Abundant TR3 mRNA expression was observed as shown in FIG. 7c (enlargement in FIG. 7d). Extensive analyses of TR3, MINOR and NOT mRNA expression was performed and the data on two specimens, derived from two distinct donors, are shown in FIG. 8 (a-f and g-l, specimen 1 and 2, respectively).

In each specimen in consecutive sections, substantial expression was observed of TR3 (a, b, g, h), MINOR (c, d, i, j) and NOT (e, f, k, 1) mRNA. In the nine different specimens analysed, most specimens showed expression of these transcription factors throughout the lesion, like in the typical examples shown, with clearly not all smooth muscle cells expressing the TR3-like factors. The percentage of cells expressing one of the nuclear receptors was determined and revealed 33±22% of the cells positive for TR3, 36±17% of the cells with NOT expression, while 17±10% expressed MINOR (Table 1). In line with the results obtained for TR3 mRNA expression, immunohistochemistry with anti-TR3 antibodies revealed that TR3 protein was also expressed in consecutive sections of these in-stent restenosis specimens (FIG. 9).

### TR3, MINOR and NOT Expression in Arteriovenous Graft Neointima in a Pig Model for AV Graft Failure

Porcine paraffin sections were analysed for the mRNA expression of TR3, MINOR and NOT by radio-active in-situ hybridization. All 3 nuclear receptors TR3, MINOR and NOT were extensively and highly expressed in the both shoulder and cushion region of the graft neointima, which mainly consists of proliferating vascular smooth muscle cells. Furthermore, TR3, MINOR and NOT were also expressed in area around the graft in which in addition of smooth muscle cells inflammatory cells like macrophages are present (FIG. 10 A-C).

### CONCLUSION

This example demonstrates the expression of all three TR3-like subfamily members in in-stent neointima and arteriovenous graft neointima. Despite the clear inhibitory effect of TR3 on smooth muscle cell growth, its activity is apparently insufficient to prevent symptomatic restenosis at sites of stent and/or graft placement in the patients studied. The present invention is based on increasing the activity of pre-existent TR3 by agonists, which intervention is aimed at diminishing neointimal formation in general and restenosis.

According to the invention it was found that C-DIMs enhance the activity of TR3-like factors. Targeting of these factors with small molecule compounds, such as C-DIMs, is highly specific for diseased areas of the vascular tree since TR3-like factors are synthesized characteristically in lesion smooth muscle cells and not in normal arteries.

### Example 3

### Agonist Activation of NUR77 (TR3) Protects Against Neointima Formation

A well-defined mouse model of neointima formation consists of placement of a non-constrictive perivascular cuff around the mouse femoral artery (Quax et al. Circulation 103: 562-569, 2001). It has been shown that the non-constrictive perivascular cuff may be constructed from a polymeric formulation suitable for controlled drug delivery (Pires et al. Biomaterials 26:5386-5394, 2005). Such a novel drug-eluting polymer cuff simultaneously induces reproducible intimal hyperplasia and allows confined delivery of drugs to the cuffed vessel segment. In the current study, these drug-eluting cuffs were applied to evaluate the local effect of TR3-agonists on neointima formation.

### Materials and Methods

### SMC Culture

Human SMCs were explanted from umbilical cord arteries. Cells were cultured in DMEM (Invitrogen Life Technology, Breda, The Netherlands) with 10% (v/v) fetal bovine serum (FBS) with penicillin and streptomycin (Invitrogen). Cells were used at passages five to seven. SMCs were characterized with a monoclonal antibody, directed against smooth muscle alpha-actin (1A4, DAKO), and demonstrated uniform fibrillar staining. To determine cellular viability, cells were washed with PBS and subsequently incubated in medium in the presence of 0.5-mg/ml 3-(4,5-dimethylthiazol-2-yl)-2,5-diphenyltetrazolium bromide (MTT; Sigma Diagnostics, St. Louis). After two hours, medium was discarded, formazan crystals were dissolved in isopropanol and optical density was measured at 590 nm. Apoptosis was induced by incubating cells for 24 hours in medium with 0.25 μM staurosporine (Sigma). Subsequently, SMCs were fixed, stained with Hoechst dye and the relative number of apoptotic nuclei was determined.

### Transfection Experiments and Luciferase Assay

Cells were electroporated using the Amaxa method(Amaxa, Germany) with nucleofector reagent for SMCs. In each transfection, 0.5−1.0×10⁶ cells were used and 3.5 μg Nur77-reporter plasmid with 1.5 μg Renilla luciferase plasmid (containing the thymidine kinase promoter) to correct for cell number and transfection efficiency. The NUR77-reporter plasmid contained the Nur response element (NurRE) of the POMC-promoter in triplicate with the −34/+63 minimal promoter of POMC gene.¹⁹ 24 hours after transfection, cells were incubated with 6-MP (Sigma) for 24 hours and luciferase activity was assayed with the Dual luciferase reporter system (Promega, Madison, Wis.).

### DNA Synthesis Assay

SMCs were seeded in 24-well plates at 1−4×10⁴ cells per well and reached 60% to 70% confluency after 24 hours. SMCs were made quiescent by incubation for 24 hours in FBS-free medium. 6-MP was dissolved in dimethylsulfoxide and applied one hour before FBS stimulation. SMCs were stimulated for 24 hours with 10% (v/v) FBS and subsequently cells were labeled for 18 hours with 0.25 μCi/well [methyl-3H]thymidine (Amersham Biosciences, Buckinghamshire, UK). Incorporated radioactivity was precipitated for 30 min at 4° C. with 10% (w/v) trichloroacetic acid, washed twice with 5% (w/v) trichloroacetic acid, and dissolved in 0.5 N NaOH (0.5 mL per well). Incorporated [³H]thymidine was measured by liquid-scintillation counting.

siRNA Experiments

The following small interfering (si)RNA sequences were used: Nur77 siRNA, 5′-CAG UCC AGC CAU GCU CCU C dTdT-3′, as described previously²⁰, and control siRNA, 5′-CAG ACG AGC CUU GCU CGU C dTdT-3′ (Ambion Inc., Austin, Tex.). Five μg of siRNA was transfected into 0.5−1×10⁶ SMCs, using Nucleofector reagent for SMCs (Amaxa) as per the manufacturer's recommendations. Total mRNA was isolated five days after transfection, using the absolutely mRNA miniprep kit (Stratagene, La Jolla, Calif.). Subsequent cDNA synthesis was performed using the iScript cDNA synthesis kit (Biorad, Hercules, Calif.). Real-time polymerase chain reaction (PCR) was performed using SYBR green mix (Biorad) in the MyIQ System (Biorad). Primers for Nur77 were as follows: (forward) 5′-GTTCTCTGGAGGTCATCCGCAAG-3′ and (reverse) 5′-GCAGGGACCTTGAGAAGGCCA-3′. As a control for equal amount of first strand cDNA in different samples we corrected for Ribosomal Phosphoprotein (P0) mRNA levels, which were determined with the following primers (forward) 5′-TCGACAATGGCAGCATCTAC-3′ and (reverse) 5′-ATCCGTCTCCACAGACAAGG-3′.

### Drug-Eluting Cuffs

Poly(ε-caprolactone)-based drug-delivery cuffs were manufactured as previously described (Pires et al. Biomaterials 26: 5386-5394, 2005). Briefly, 6-MP was dissolved at different concentrations in blended, molten drug-polymer mix and cuffs were designed to fit around the femoral artery of mice. Drug-eluting cuffs are shaped as longitudinally cut cylinders with an internal diameter of 0.5 mm, an external diameter of 1.0 mm, a length of 2.0 mm and a weight of approximately 5.0 mg. Drug-eluting cuffs were loaded with 0.5% (w/w) and 1% (w/w) 6-MP and the in vitro release profiles were determined for a 4-week period, as described before (n=5/group). 6-MP showed a sustained and dose-dependent release. Total release at 4 weeks was: 11.3±2.3 μg (46.3%) and 30.0±3.5 μg (58.7%) for the 0.5% and 1% 6-MP-eluting cuff, respectively.

### Femoral Artery Cuff Murine Model

All animal work was approved by AMC institutional regulatory authority and carried out in compliance with guidelines issued by the Dutch government. Wild-type FVB mice (Wt), transgenic mice expressing the full-length Nur77 gene (Nur77), or mice expressing a dominant-negative variant of Nur77 (ΔTA) (the latter two strains under control of the SM22α promoter, which directs the expression of transgenes specific to SMCs), in an FVB background, were used for experiments. Male mice, 10-12 weeks old, were fed a standard chow diet. At the time of surgery, mice were anaesthetized with an intraperitoneal injection of 5 mg/kg Dormicum (Roche, Basel, Switzerland), 0.5 mg/kg Dormitor (Orion, Helsinki, Finland) and 0.05 mg/kg Fentanyl (Janssen, Geel, Belgium). The femoral artery was dissected from its surroundings and loosely sheathed with a non-constrictive cuff. Either a control, empty cuff or a 6-MP eluting cuff (0.5% or 1% w/w) was used (n=6/group).

Nur77 (TR3) mRNA Expression in Cuffed Mouse Femoral Artery

Male Wt mice underwent femoral artery cuff placement as described above. Animals were sacrificed at different timepoints after surgery (0, 6, 24, 48, 72 hours, and 7 days), employing four mice for each timepoint. Femoral arteries were isolated, harvested and snap frozen. Total RNA was isolated using the RNeasy Fibrous Tissue Mini-Kit (Qiagen, Venlo, The Netherlands), according to the manufacturer's protocol. cDNA was made from all RNA samples, using Ready-To-Go RT-PCR beads (Amersham Biosciences, Uppsala, Sweden).

Intron-spanning primers and probes were designed to hybridize with murine Nur77 cDNA (sense: 5′-GGGCATGGTGAAGGAAGTTGT-3′; antisense: 5′-AGGCTGCTTGGGTTTTGAAG-3′; Probe: 5′-CCGCCCTTTTAGGCTGTC TGTCCG-3′), using Primer Express™ 1.5 (Applied Biosystems, Foster City, Calif.). Hypoxanthine phosphoribosyltransferase (HPRT) was assayed to correct for cDNA input. For each timepoint, RT-PCR was performed in duplicate. Data are presented as fold induction of Nur77 mRNA expression in injured over non-injured vessels.

### Quantification and Histological Assessment of Intimal Lesions in Cuffed Femoral Arteries

Wt and Nur77-overexpressing transgenic mice were sacrificed at 28 days after cuff placement, whereas ATA transgenic mice were sacrificed at 14 days after surgery. The thorax was opened and a mild pressure-perfusion (100 mmHg) with 4% (v/v) formaldehyde in 0.9% (w/v) NaCl was performed for 5 min by cardiac puncture. After perfusion, femoral arteries were harvested, fixed overnight in 4% (v/v) formaldehyde, dehydrated and paraffin embedded. Serial cross-sections (5 μm thick) for histological analysis were used throughout the entire length of the cuffed femoral artery. All samples were routinely stained with hematoxylin-phloxine-saffron (HPS). Weigert's elastin staining was used to visualize elastic laminae. Ten equally spaced cross-sections were used in all mice to quantify intimal lesions. Using image analysis software (Leica Qwin, Wetzlar, Germany), total cross sectional medial area was measured between the external and internal elastic lamina; total cross sectional intimal area was measured between the endothelial cell monolayer and the internal elastic lamina.

### Statistical Analysis

In vitro experiments were repeated at least twice and are presented as mean±SD and were analyzed by the Student t-test. Animal experiments are presented as mean±SEM and were analyzed using the Mann-Whitney U-test (SPSS 11.5 for Windows). P-values less than 0.05 were regarded as statistically significant.

### Results

### 6-MP Enhances Nur77 (TR3) Activity in Cultured SMCs

To investigate whether 6-MP increases Nur77 transcriptional activity in vascular cells, human SMCs were transduced with lentivirus encoding Nur77, resulting in expression in 85-90% of the infected cells and increased Nur77 mRNA levels compared to mock-infected SMCs (data not shown). Immunofluorescence of transduced SMCs revealed Nur77 protein overexpression located to the nucleus (data not shown). Nur77-overexpressing SMCs were subsequently electroporated with the firefly luciferase reporter construct, containing the palindromic NurRE (Nur77 response element from the POMC-promoter) sequence (TGATATTTn₆AAATGCCA) to monitor Nur77 transcriptional activity in combination with the thymidine kinase-renilla luciferase construct as a control for transfection efficiency.

Incubation of SMCs for 24 hours with 50 μM 6-MP resulted in a 10-fold increase in Nur77 activity (FIG. 11). These data clearly indicate that the agonist 6-MP robustly enhances Nur77 (TR3) activity in cultured SMCs.

### 6-MP Inhibits Proliferation of SMCs: Involvement of Nur77 (TR3)

To study whether 6-MP modulates SMC proliferation, we investigated DNA synthesis of cultured, human SMCs in the presence of increasing concentrations of 6-MP. As expected, 6-MP inhibits DNA synthesis in SMCs (FIG. 12A). To assess the specific contribution of Nur77 in this process, Nur77 expression was knocked down by small interfering (si)RNA in human SMCs. Transfection with siRNA directed against Nur77 or with control siRNA, results in downregulation of FBS-induced Nur77 mRNA levels in the siNur77 transfected cells, as determined by real-time RT-PCR (FIG. 12B). [³H]Thymidine incorporation is significantly higher in cells in which Nur77 is knocked down by gene-specific siRNA in comparison to SMCs transfected with control siRNA. To visualize the relative effect of 6-MP on DNA synthesis in SMCs transfected with siNur77 RNA or with control siRNA, we expressed [³H]Thymidine incorporation as percentage of control condition (FIG. 12C). DNA synthesis is inhibited by 6-MP for 61% when SMCs are transfected with control siRNA, whereas the effect of 6-MP is significantly less in SMCs transfected with Nur77-specific siRNA, since only 41% inhibition of DNA synthesis is observed. These data clearly demonstrate that 6-MP inhibits DNA synthesis in SMCs at least partly through activation of Nur77. Possibly, the remaining effect of 6-MP may be attributed to residual Nur77 activity and/or to 6-MP-mediated-Nurr1 and/or NOR-1 activation.

6-MP is not Cytotoxic to SMCs and does not Induce Apoptosis

To verify whether 6-MP is cytotoxic, quiescent SMCs were incubated for 24 hours with increasing concentrations of 6-MP and viability of the cells was determined with a standard MTT assay. The number of viable cells is reduced in response to staurosporine (35% reduction), whereas 6-MP does not affect cellular viability (FIG. 13A), indicating that under these conditions 6-MP has no cytotoxic effect on human SMCs.

To investigate whether 6-MP induces apoptosis in SMCs, quiescent SMCs were incubated for 24 hours with increasing concentrations 6-MP. As a positive control, staurosporine was shown to induce apoptosis in SMCs (50%±4%). Clearly, no evidence was found that 6-MP affects cell death under these conditions (3%±2%) in comparison to control cells (4%±2%). (FIG. 13B).

### Nur77 (TR3) is Expressed During the Process of Neointima Formation

To assess the potential inhibitory effect of the NUR77 (TR3) agonist 6-MP on SMC-rich lesion formation in vivo, the well-established murine model of cuff-induced neointima formation was applied. Nur77 mRNA expression was studied during neointima formation and, as depicted in FIG. 14, Nur77 mRNA expression is upregulated after cuff placement as a function of time and shows optimal expression 6 hours after vascular injury (189±26-fold increase). Nur77 mRNA expression is enhanced up to 7 days after surgery in comparison to non-cuffed sham-operated vessels (13.4±1.1-fold increase). Given that Nur77 mRNA transcripts are regulated upon vascular injury strictly dependent on conditions and time, it is conceivable that Nur77 plays a role in the process of neointima formation.

### Effect of 6-MP on Cuff-Induced Neointima Formation in Wild-Type, Nur77 and ΔTA Transgenic Mice

To evaluate the effect of 6-MP on cuff-induced neointima formation in vivo, a drug-eluting cuff was employed loaded with increasing concentrations of 6-MP, which allows restricted, local perivascular delivery of compounds to the cuffed vessel segment. The effect of 6-MP was initially evaluated in wild-type (Wt) animals and transgenic mice expressing full-length Nur77 cDNA in the arterial vessel wall. Microscopic analysis of cuffed femoral artery segments revealed that, after four weeks, a concentric neointima was formed in mice receiving a control empty drug-eluting cuff in both Wt and Nur77-transgenic mice. Animals receiving a 6-MP-eluting cuff showed reduced intimal hyperplasia (FIG. 15A).

Morphometric analyses revealed significant inhibition of cuff-induced neointima formation in vessel segments, locally treated with the higher (1%) 6-MP concentration, in both Wt (P=0.02) and Nur77 transgenic mice (P=0.007, FIG. 15B). Wt animals treated with 0.5% 6-MP-eluting cuffs did not show a decrease in neointima formation (P=0.32), while the same 6-MP concentration substantially reduced neointima formation in Nur77 transgenic mice (P=0.02). No changes were observed in medial areas of the cuffed femoral arteries (data not shown).

Consequently, a similar dose-dependent decrease was seen in intima/media ratios of 6-MP-treated Nur77 transgenic mice; control cuff: 0.75±0.11; 0.5% 6-MP: 0.47±0.05, (P=0.04); 1% 6-MP: 0.30±0.06, (P=0.003). Again, intima/media ratios of cuffed arteries in Wt mice were only significantly decreased in the 1% 6-MP cuffs: control cuff: 1.10±0.16; 0.5%: 0.75±0.05, (P=0.15); 1%:0.51±0.03, (P=0.008).

To further establish functional involvement of Nur77 in 6-MP-mediated effects on neointima formation, 6-MP-eluting cuffs were placed around the femoral artery of transgenic mice, expressing a dominant-negative variant of Nur77 (ATA) that inhibits the activity of all three Nur77-like factors.

Previously, it was shown that SMC-rich lesions develop relatively fast after carotid artery ligation in ΔTA transgenic mice. In line with these data, in this study enhanced lesion formation in the currently applied femoral artery cuff model was observed, resulting in almost fully occlusive lesions within 4 weeks (data not shown). To reliably evaluate the effect of 6-MP-eluting cuffs in ATA transgenic mice, we analyzed neointima formation after 2 weeks (FIG. 15A). Morphometric analyses of intimal area revealed that local delivery of 6-MP in ATA transgenic mice did not change media thickness and had no significant effect on neointima formation neither in the 0.5% (P=0.46) nor in the 1% (P=0.37) 6-MP cuff (FIG. 15C).

Altogether, these data are in line with the in vitro observations and demonstrate that 6-MP inhibits cuff-induced neointima formation involving activation of Nur77.

It has thus been shown that enhancing the activity of NUR77 (TR3) by 6-MP inhibits SMC proliferation and protects against SMC-rich lesion formation. These observations clearly show that the nuclear receptor Nur77 (TR3) is a potential target to prevent (in-stent) restenosis.

### CONCLUSION

In conclusion, it has been demonstrated that Nur77 is highly expressed in mice during cuff-induced neointima formation, but not in murine sham-operated arteries.

Furthermore, it has been clearly shown that activation of Nur77 by 6-MP reduces human SMC proliferation and protects against neointima formation in a mouse restenosis model. Activation of the nuclear receptor Nur77 by 6-MP or by other activators/agonists thus is a rational approach to treat (in-stent) restenosis.

### Example 4

### C-DIM-Mediated Activation of TR3 Protects Against Excessive Smooth Muscle Cell Proliferation and Smooth Muscle Cell-Rich Lesion Formation in Mice

In this example, it is shown that C-DIM derivatives inhibit SMC proliferation in vitro and the in vivo application is described of C-DIMs in a validated mouse restenosis mouse model with drug-eluting cuffs. In the mouse model as described in Moroi M et al., (J. Clin. Invest. (1998) 101:1225-32) a loosely-fitting perivascular cuff around the femoral artery induces the formation of a smooth muscle cell-rich lesion, which resembles smooth muscle cell-specific pathologies observed in humans. This model has been adapted into drug-eluting cuffs, reminiscent to drug-eluting stents. C-DIMs are reversibly attached to these cuffs and the effect in lesion formation is evaluated by operational procedures. This experiment was performed with wild-type, TR3 and dTA transgenic mice. Extensive knowledge on the pharmacology and toxicity of C-DIM derivatives is available.

### Materials and Methods

### SMC Culture

Human SMCs were explanted from umbilical cord arteries. Cells were cultured in DMEM (Invitrogen Life Technology, Breda, The Netherlands) with 10% (v/v) fetal bovine serum (FBS) with penicillin and streptomycin (Invitrogen). Cells were used at passages five to seven. SMCs were characterized with a monoclonal antibody, directed against smooth muscle alpha-actin (1A4, DAKO), and demonstrated uniform fibrillar staining. To determine cellular viability, cells were washed with PBS and subsequently incubated in medium in the presence of 0.5-mg/ml 3-(4,5-dimethylthiazol-2-yl)-2,5-diphenyltetrazolium bromide (MTT; Sigma Diagnostics, St. Louis). After two hours, medium was discarded, formazan crystals were dissolved in isopropanol and optical density was measured at 590 nm. Apoptosis was induced by incubating cells for 24 hours in medium with 0.25 μM staurosporine (Sigma). Subsequently, SMCs were fixed, stained with Hoechst dye and the relative number of apoptotic nuclei was determined.

### DNA Synthesis Assay

SMCs were seeded in 24-well plates at 1−4×10⁴ cells per well and reached 60% to 70% confluency after 24 hours. SMCs were made quiescent by incubation for 24 hours in FBS-free medium. 6-MP was dissolved in dimethylsulfoxide and applied one hour before FBS stimulation. SMCs were stimulated for 24 hours with 10% (v/v) FBS and subsequently cells were labeled for 18 hours with 0.25 μCi/well [methyl-3H]thymidine (Amersham Biosciences, Buckinghamshire, UK). Incorporated radioactivity was precipitated for 30 min at 4° C. with 10% (w/v) trichloroacetic acid, washed twice with 5% (w/v) trichloroacetic acid, and dissolved in 0.5 N NaOH (0.5 mL per well). Incorporated [³H]thymidine was measured by liquid-scintillation counting.

siRNA Experiments

The following small interfering (si)RNA sequences were used: Nur77 siRNA, 5′-CAG UCC AGC CAU GCU CCU C dTdT-3′, as described previously²⁰, and control siRNA, 5′-CAG ACG AGC CUU GCU CGU C dTdT-3′ (Ambion Inc., Austin, Tex.). Five μg of siRNA was transfected into 0.5−1×10⁶ SMCs, using Nucleofector reagent for SMCs (Amaxa) as per the manufacturer's recommendations. Total mRNA was isolated five days after transfection, using the absolutely mRNA miniprep kit (Stratagene, La Jolla, Calif.). Subsequent cDNA synthesis was performed using the iscript cDNA synthesis kit (Biorad, Hercules, Calif.). Real-time polymerase chain reaction (PCR) was performed using SYBR green mix (Biorad) in the MyIQ System (Biorad). Primers for Nur77 were as follows: (forward) 5′-GTTCTCTGGAGGTCATCCGCAAG-3′ and (reverse) 5′-GCAGGGACCTTGAGAAGGCCA-3′. As a control for equal amount of first strand cDNA in different samples we corrected for Ribosomal Phosphoprotein (P0) mRNA levels, which were determined with the following primers (forward) 5′-TCGACAATGGCAGCATCTAC-3′ and (reverse) 5′-ATCCGTCTCCACAGACAAGG-3′.

### Drug-Eluting Cuffs

C-DIM eluting cuffs were made by mixing C-DIM derivatives at 70° C. with polycaprolactene and casting a tubing (0.5 mm inner diameter, 1.0 mm outer diameter). Described in detail in Pires et al., Biomaterials. 2005; 26:5386-94.

### Femoral Artery Cuff Placement

All animal work was approved by AMC institutional regulatory authority and carried out in compliance with guidelines issued by the Dutch government. Wild-type FVB mice (Wt), transgenic mice expressing the full-length Nur77 gene (Nur77), or mice expressing a dominant-negative variant of Nur77 (ΔTA) (the latter two strains under control of the SM22α promoter, which directs the expression of transgenes specific to SMCs), in an FVB background, were used for experiments. Male mice are anaesthetized with an intraperitoneal injection with a solution of Midazolam (12.5 mg/kg bodyweight) and Hypnorm (0.01 ml/mouse). The left femoral artery is isolated from surrounding tissue, loosely sheathed with a 2.0-mm cuff made of polycaprolactene and polyethylene glycol 0.5 mm inner diameter, 1.0 mm outer diameter was placed loosely around the femoral artery and tied in place with a 6-0 suture. The cuff is wider than the vessel and does not obstruct blood flow. The right femoral artery was dissected from surrounding tissue (sham-operated), but a cuff was not placed. The femoral arteries were replaced, and the wounds were sutured. After recovery from anaesthesia, the animals were given standard diet and water ad libitum. Wild-type mice and TR3- or dTA-transgenic mice are either treated with bare, control cuffs or with C-DIM-eluting cuffs, in each group 6 mice are included.

### Histological Assessment of Intimal Lesions

After 2 to 4 weeks mice were anaesthetized, the thorax was opened and mild pressure-perfusion (100 mmHg) with 3.7% formaldehyde in 0.9% NaCl (wt/vol) for 10 min was performed by cardiac punctures. After perfusion, the femoral artery was harvested, fixed overnight and paraffin-embedded. Serial sections (5 mm thick) were used throughout the entire length of the cuffed femoral artery for histological analysis.

### Morphological Quantification in Sections of Cuffed Femoral Artery

Paraffin sections are stained with haematoxilin/eosin and ten equally spaced (200 mm) cross sections are used to quantify intimal lesion. Using image analysis software (Leica, Qwin) total cross-sectional medial area are measured between the external and internal elastic lamina. Total cross-sectional intimal area is also measured between the endothelial cell monolayer and the internal elastic lamina.

### Statical Analysis

Statistical analyses were performed with SPSS, version 10.0.5 software. Experimental values are expresses as mean SEM. The significance of differences was determined by using the nonparametric Mann-Whitney 2-tailed U test and expressed as a probability value.

### Results

### C-DIM-Derivatives Inhibit Proliferation of SMCS.

To study whether C-DIMs modulate SMC proliferation, we investigated DNA synthesis of cultured, human SMCs in the presence of increasing concentrations of C-DIM-H, C-DIM-OCH3 or C-DIM-CF₃ by [³H]thymidine icorporation assays. All three C-DIMs inhibit DNA synthesis in SMCs dose dependently (FIG. 16). C-DIM-His less effective than C-DIM-OCH3 and C-DIM-CF₃.

### C-DIM-Derivatives and Smooth Muscle Cell Viability.

To verify whether C-DIMs are cytotoxic, quiescent SMCs were incubated for 24 hours with increasing concentrations of C-DIM compounds and viability of the cells was determined with a standard MTT assay. C-DIM-H and C-DIM-OCH3 reduced cell viability of the cells at 20 μM moderately, whereas up to 10 μM no significant reduction in cell viability was observed for C-DIM-H and C-DIM-OCH3 (FIG. 17). To investigate whether C-DIMs induce apoptosis in SMCs, quiescent SMCs were incubated for 24 hours with C-DIMs at a final concentration of 10 μM. As a positive control, staurosporine was shown to induce apoptosis in SMCs. Clearly, no evidence was found that C-DIMs affect cell death under these conditions in comparison to control cells (FIG. 18).

### Inhibition of SMC Proliferation by C-DIM-OCH3 Involves TR3.

To assess the specific contribution of TR3 in C-DIM-OCH3 mediated inhibition of SMC proliferation, TR3 expression was knocked down by small interfering (si)RNA in human SMCs. Transfection with siRNA directed against TR3 or with control siRNA, results in downregulation of FBS-induced TR3 mRNA levels in the siTR3 transfected cells, as determined by real-time RT-PCR (FIG. 12B). To reveal the relative effect of C-DIM-OCH3 on DNA synthesis in SMCs transfected with siTR3RNA or with control siRNA, we expressed [³H]Thymidine incorporation as percentage of control condition (FIG. 19). DNA synthesis is inhibited by C-DIM-OCH3 for 49.7% when SMCs are transfected with control siRNA, whereas the effect of C-DIM-OCH3 is significantly less in SMCs transfected with TR3-specific siRNA, since only 40.1% inhibition of DNA synthesis is observed. These data clearly demonstrate that C-DIM-OCH3 inhibits DNA synthesis in SMCs at least partly through activation of TR3. Most likely, also Nurr1 and possibly also NOR-1 are involved in C-DIM-OCH3-mediated effects, explaining why no full inhibition of the C-DIM-OCH3 effect was observed after TR3 knockdown.

### Effect of C-DIMs on Cuff-Induced Smooth Muscle Cell-Rich Lesion Formation in Wild-Type, TR3 and ΔTA-Transgenic Mice.

From the mice treated with bare, control cuffs the TR3 mice showed less smooth muscle cell-rich lesion formation in the left femoral artery than wild-type mice. The dTA mice developed larger smooth muscle cell-rich lesions than wild-type mice, which is in line with the knowledge that dTA is a dominant-negative inhibitor of TR3, MINOR and NOT. TR3-mice treated with C-DIM-eluting cuffs developed again smaller lesions than the bare cuff treated TR3 mice. Also in wild-type mice the lesion size was smaller due to incorporation of DIM in the cuff. In contrast, the extent of lesion formation in dTA mice was similar for bare cuffs and C-DIM-eluting cuffs. The latter result was expected, because in the dTA mice the activity of endogenous as well as transgenic TR3-like factors is blocked. These data clearly indicate that C-DIM derivatives inhibits the formation of smooth muscle cell-rich lesions in a TR3-like factor dependent way.

### Example 5

### Nuclear Receptors Nur77, Nurr1 and NOR-1 Expressed in Atherosclerotic Lesion Macrophages Reduce Lipid Loading and Inflammatory Responses

In this example, it is shown for the first time that of all three NR4A family members Nur77, Nurr1 and NOR-1 are expressed in human atherosclerotic lesion macrophages and it is demonstrated that these factors reduce the uptake of oxidized low-density lipoprotein (ox-LDL) as Well as inhibit the inflammatory response to inflammatory stimuli in human macrophages.

### Materials and Methods

### Human Tissue Specimens

Human tissue samples were obtained with informed consent from organ donors, according to protocols approved by the Medical Ethics Committee of the Academic Medical Center, Amsterdam. The specimens were paraffin embedded, sectioned, and mounted on glass slides (Superfrost-Plus, Emergo).

Vascular specimens were characterized by immunohistochemistry with antibodies specific for SMCs and macrophages to establish the stage of disease according to the American Heart Association classification.

### Immunohistochemistry and Double In-Situ Immunohistochemistry

Macrophages were detected by the monoclonal antibody Ham56 (DAKO) and SMCs by the monoclonal antibody 1A4 (DAKO) directed against smooth muscle α-actin, in human vascular specimens. Anti-Nur77 (M-210, Santa Cruz Biotechnology), anti-Nurr1 (M-196, Santa Cruz Biotechnology) and anti-NOR-1 were used to detect the NR4A nuclear receptors. Briefly, after deparaffinization and endogenous peroxidase quenching, citrate antigen retrieval was performed, followed by blocking and permeabilization with 1% (w/vol) bovine serum albumin, 1% (vol/vol) normal goat serum and 0.5% Triton-X 100 and primary antibody incubation overnight at 4° C. After biotin-labeled goat-anti-rabbit IgG secondary antibody (DAKO) incubation followed by steptavidin-HRP (DAKO), AEC (Sigma) detection was applied. Staining after secondary antibody incubation alone served as a negative control.

Combination of radioactive gene-specific in situ hybridization and macrophage-specific immunohistochemistry was essentially performed as described previously. For in situ hybridization the following riboprobes were synthesized: Nur77, GenBank No. L13740, bp 1221 to 1905; Nurr1, GenBank No. X75918, bp 119 to 1003 and NOR-1, GenBank No. U12767, bp 1435 to 2172. After hybridization macrophages were detected using immunohistochemistry as described above, followed by emulsion radiography. Matching sense riboprobes were assayed for each gene and were shown to give neither background nor a non-specific signal. The sections were exposed for 4 to 8 weeks. All slides were counterstained with hemotoxylin and embedded in glycergel (DAKO).

### Cell Culture

Primary human monocytes/macrophages were isolated from buffy-coats of blood donors, obtained from the Dutch central bloodbank Sanquin. After isolation by Ficoll-Paque (Pharmacia Biotech) gradient centrifugation, monocyte-negative selection kit (Dynal) and adhesion-mediated purification, cells were cultured for 48 hours at a density of 0.5-1×10⁶ cells/ml before experiments were performed. Human monocytic THP-1 cells (ATCC) were cultured in RPMI 1640, 10% (vol/vol) fetal bovine serum and 100 U/ml penicillin/streptomycin (GIBCO-BRL). Cells were plated in 12-wells plates at a density of 0.5×10⁶ cells/ml, differentiated into macrophages by PMA (100 ng/ml) for 48 hours. After differentiation, cells were washed twice with PBS and grown in medium for 24 hours. Reagents used were PMA (Sigma), LPS (Sigma), recombinant human TNFα (R&D) and DiI-labeled ox-LDL (Intracel-RP-173).

### Lentivial Vector Construction and Production

hNur77 cDNA (GenBank D49728, bp 8-1920) was cloned into the XbaI-NdeI sites of the pRR1-cPPt-PGK-PreSIN vector (PGK-Nur77). hNurr1 cDNA (Genbank X75918, bp 73-2310) was placed into the SalI-NsiI sites of the pRR1-cPPt-PGK-PreSIN vector (PGK-Nurr1) and hNOR-1 cDNA (Genbank D78579, bp 513-2872) was ligated into the XbaI site of the pRR1-cPPt-PGK-PreSIN vector (PGK-NOR-1). PGK-EGFP-PreSIN (PGK-EGFP) was constructed by isolating the EGFP cDNA from the expression vector pEGFP-N2 (Clontech) using SalI-XbaI digestion, subsequently ligated into the corresponding sites of the pRR1-cPPt-PGK-PreSIN vector. All constructs were verified by DNA sequencing. Virus stocks were produced as known. Briefly, 20 μg of PGK transfer vector, 13 μg of pMDLg/pRRE, 7 μg pVSV-g, and 5 μg of pRSV-REV were co-transfected into 180 cm² HEK293T cells using the calcium phosphate co-precipitation method. Conditioned medium was harvested at 48 hours and 72 hours after transfection, filtered through 0.45 μm filters and concentrated by ultra centrifugation (20.000 rpm, 2 hours, 4° C.). Determination of viral titers was performed by transducing HEK 293 cells with serially diluted viral concentrate, 48 hours after transduction total genomic DNA was isolated from these cells and the number of vector DNA copies was determined using PCR analysis with pRR1-cPPt-PGK-PreSIN vector as calibration standard (forward primer: 5′-GTGCAGCAGCAGAACAATTTG-3′, reverse primer: 5′-CCCCAGACTGTGAGTTGCAA-3′).

### Lentiviral Infection

THP-1 cells were transduced in the presence of 10 μgr/ml DEAE-dextran with recombinant lentivirus for 24 hours at a Multiplicity of infection of 3. Empty (Mock) and EGFP lentivirus were taken along as controls. After transduction cells were cultured in suspension for 72 hours, differentiated into macrophages and cultured as described above. Overexpression of Nur77, Nurr1, NOR-1 and EGFP was checked by flow cytometric analyses (EGFP) and immunofluorescence (FIG. 16). Briefly, cells cultured on glass were fixed for 20 min with 4% (w/vol) paraformaldehyde PBS and permeabilized with 0.5% (vol/vol) Triton-X-100. Cells were stained by anti-Nur77 (M-210, Santa Cruz Biotechnology), anti-Nurr1 (M-196, Santa Cruz Biotechnology) and anti-NOR-1 for detection of Nur77, Nurr1 and NOR-1 respectively, followed by Alexa Fluor 488-conjugated goat anti-rabbit IgG or Alexa Fluor 568-conjugated donkey anti-goat IgG (Molecular Probes). Nuclei were stained with Hoechst.

### RNA and Protein Analysis

Total RNA was extracted using RNA absolutely Miniprep kit (Stratagene). cDNA was made using iScript cDNA Synthesis kit (Biorad) and semi-quantitative real-time RT-PCR was performed using iQ SYBR-Green Super-Mix in the MyiQ RT-PCR system (Biorad). Specific primers for Nur77, Nurr1, NOR-1, scavenger receptor-A (SR-A), CD36, macrophage inflammatory protein-1a (MIP-1α) and -1β (MIP-1β), MCP-1, IL-8, IL-1β, IL-6 and ribosomal protein P0 were designed as follows;

Nur77:
Fw: 5′-gttctctggaggtcatccgca ag-3′
Rv: 5′-gcagggaccttgagaaggcca-3′
Nurr1:
Fw: 5′-tattccaggttccaggcgaa-3′
Rv: 5′-gctaatcgaaggacaaacag-3′
NOR-1:
Fw: 5′-cc aagccttagcctgcctgtc-3′
Rv: 5′-agcctgtcccttact ctggtgg-3′
IL-1β:
Fw: 5′-tggcagaaagggaaca gaaagg-3′
Rv: 5′-gtgagtaggagaggtgagagagg-3′
IL-6:
Fw: 5′-tgtagccgccccacacag-3′
Rv: 5′-gctg ctttcacacatgttactcttg-3′
IL8:
Fw: 5′-ctgcg ccaacacagaaatta-3′
Rv: 5′-attgcatctggcaacc ctac-3′
MIP-1β:
Fw: 5′-gcgtgactgtcctgtctctcc-3′
Rv: 5′-accacaaagttgcgaggaagc-3′
MIP-1α:
Fw: 5′-acgggcagcagacagtgg-3′
Rv: 5′-ggcgtgtcag cagcaagtg-3′
MCP-1:
Fw: 5′-cctagcttt ccccagacacc-3′
Rv: 5′-cccaggggtagaactgtgg-3′
SR-A:
Fw: 5′-ctcgctcaatgacagctttgcttc-3
Rv: 5′-tcgtttcccacttcaggagttgag-3′
CD36:
Fw: 5′-gagaactgttatggggctat-3′
Rv: 5′-ttcaactggagaggc aaagg-3′
P0:
Fw: 5′-tcgacaatggcagc atctac-3′
Rv: 5′-atccgtctccacagacaagg-3′

All RT-PCR data were corrected for housekeeping gene ribosomal protein P0. Protein levels of IL-8, IL-1β and IL-6 were determined in supernatant of cell cultures by BD™ Cytometric Bead Array according to manufacturers' protocol. Experiments were performed in duplicate and repeated at least twice.

### Lipid Loading, Quantification and Microscopy

After lentiviral infection THP-1-derived macrophages were treated with DiI-labeled ox-LDL for time periods indicated, subsequently washed twice with PBS and lysed in pure isopropanol. After sonification and 10 minutes centrifugation (13000 g) supernatant was measured by fluorometry. For confocal microscopy, cells were cultured on glass and treated with DiI-labeled ox-LDL. Experiments were performed in duplicate and repeated at least twice.

### Statistical Analyses

Student's t-tests were performed. Fold inductions and percentages were calculated after normalization to the control.

### Results

### Nur77, Nurr1 and NOR-1 are Expressed in Human Atherosclerotic Lesion Macrophages

In previous studies expression of Nur77, Nurr1 and NOR-1 in both SMCs and ECs in atherosclerotic lesions was demonstrated. In this study, expression of Nur77, Nurr1 and NOR-1 in atherosclerotic lesion macrophages was demonstrated by combining macrophage-specific immunostaining with gene-specific in-situ-hybridization. Aorta specimens of 8 different organ donors (3 males and 5 females, age 40-69 years) were characterized by immunohistochemistry according to the American Heart Association guidelines (Table 2; FIG. 20A, B and 21 A, B). The complexity of the lesions analyzed ranged from class II to VI. mRNA expression levels of Nur77, Nurr1 and NOR-1 in lesion macrophages and SMCs were scored and specific localization of expression in the lesion indicated. As a typical example of an early lesion, a type II lesion with high mRNA expression levels of all three nuclear receptors in macrophages is shown (FIG. 20, C—H; ‡ in Table 2). Protein expression of Nur77, Nurr1 and NOR-1 localizes to the nucleus in macrophage-rich areas and is comparable with the mRNA expression pattern (FIG. 21, A-E; ‡ in Table 2). Notably, in complex lesions, prominent macrophage-specific expression is localized to distinct lesion areas, especially to shoulder regions and macrophages infiltrated in the media.

### Activated Primary Human Macrophages and THP-1-Derived Macrophages Express Nur77, Nurr1 and NOR-1.

High expression levels of the NR4A factors in atherosclerotic lesion macrophages prompted us to study whether their expression is dependent on inflammatory conditions that are usually encountered at these diseased areas. In addition, the functional activity of these transcription factors was determined in in vitro studies. The expression of Nur77, Nurr1 and NOR-1 in both primary monocytes/macrophages as well as in PMA-treated THP-1-derived macrophages was assayed by semi-quantitative real-time RT-PCR and immunofluorescence in response to inflammatory stimuli. In primary monocytes/macrophages (derived from 2 different donors) mRNA expression levels of all three nuclear receptors are highly induced by LPS and moderately induced by TNFα2 hours after stimulation (FIG. 22A). Similarly, in THP-1-derived macrophages Nur77, Nurr1 and NOR-1 are strongly induced (50-150 fold) in response to LPS, 2 hours after stimulation and low-to-moderately induced (3-6 fold) in response to TNFα. Nur77 and Nurr1 expression is optimal at 1 hour, whereas NOR-1 mRNA induction is optimal 3 hours after TNFα stimulation (FIG. 22B). Time course mRNA expression curves were performed and showed transient induction of all three transcription factors in response to both LPS and TNFα stimulation (FIG. 22C). Immunofluorescence analysis revealed enhanced NOR-1 protein expression 6 hours after LPS stimulation localizing to the nucleus (FIG. 22D).

Lentiviral Overexpression of Nur77, Nurr1 and NOR-1 Reduces ox-LDL Lipid Loading.

To study the function of Nur77, Nurr1 and NOR-1 in macrophages, THP-1 cells were infected with lentiviruses that express these factors or control Mock-virus and determined the effect on lipid loading, a hallmark of atherosclerosis. Nur77, Nurr1 and NOR-1 overexpressed by recombinant lentivirus resulted in 80-90% transduction efficiency and nuclear localization of the encoded proteins (FIG. 23). The uptake of DiI-labeled ox-LDL was quantified by fluorometry. In macrophages overexpressing NR4A factors there is a trend of reduced lipid uptake already after 3 to 6 hours, with a more than 30% reduction after 24 hours (FIG. 24A). Confocal microscopy was performed to assess the cellular localization of DiI-labeled ox-LDL in macrophages. After 24 hours DiI-fluorescence localizes to lipid vacuoles and fluorescence intensity is low in Nur77-overexpressing macrophages as compared to Mock-lentivirus infected cells (FIG. 24B).

Since SR-A and CD36 are important genes involved in modified lipoprotein uptake, mRNA expression levels of these genes were determined by semi-quantitative real-time RT-PCR. THP-1 macrophages overexpressing Nur77, Nurr1 and NOR-1 express significantly lower levels of SR-A and CD36 than Mock-virus infected cells (FIG. 24C).

### Lentiviral Overexpression of Nur77, Nurr1 and NOR-1 Reduces Inflammatory Chemokine and Cytokine Expression

Next, the effect of lentivirus-mediated overexpression of Nur77, Nurr1 and NOR-1 on chemokine- and cytokine-mRNA expression and secreted protein concentration was assayed(FIG. 25). mRNA levels of the chemokines MIP-1α and -1β, MCP-1 and IL-8 and of the pro-inflammatory cytokines IL-1β, and IL-6 were determined by semi-quantitative real-time RT-PCR after stimulation with LPS, TNFα or vehicle (FIG. 25A). As a control for the activity of LPS and TNFα, mRNA levels were assayed in Mock-infected macrophages (FIG. 25A). Except for IL-6 expression, which is not detectable (ND) after TNFα stimulation, mRNA expression levels of these inflammatory genes are induced 20-8000 fold by LPS and 3-10 fold by TNFα. mRNA levels of these chemokines and cytokines analyzed are robustly reduced (2-10 fold) in THP-1-macrophages overexpressing either Nur77, Nurr1 or NOR-1 as compared to Mock-infected cells both after LPS and TNFα stimulation, as well as in their unstimulated controls. As an exception, MCP-1 mRNA expression is 2.5 fold induced by TNFα in NOR-1 overexpressing macrophages and not significantly different in Nurr1 overexpressing cells as compared to Mock-infected cells. In addition to the mRNA results described, we determined protein concentrations of IL-8, IL-1β and IL-6 (FIG. 25B) in the conditioned medium of lentivirus-infected THP-1 macrophages. Conditioned media were collected at 0, 6 and 24 hours after treatment with LPS and protein concentrations were determined by BD™ Cytometric Bead Array.

Overexpression of Nur77, Nurr1 or NOR-1 results in a significant reduction of LPS induced secretion of IL-8, IL-1p and IL-6 by THP-1 macrophages, except for IL-8 in case of NOR-1 overexpression.

TABLE 2
Donor characteristics and mRNA expression profiles of Nur77, Nurr1 and NOR-1

| | | | | | | | | | Area of |
|---|---|---|---|---|---|---|---|---|---|
| | Age | Class | Lesion MØ | | | Lesion SMC | | | expression in |
| Sex | (yrs) | (AHA) | Nur77 | Nurr1 | NOR-1 | Nur77 | Nurr1 | NOR-1 | vessel wall |
| F | 40 | II | + | ++ | ++ | ++ | + | + | neointima |
| F | 41 | II | + | + | + | ++ | + | + | neointima |
| F^{‡} | 56 | II | ++ | ++ | ++ | ++ | + | + | neointima |
| F | 59 | II | + | ++ | ++ | ++ | + | + | neointima |
| M^{†} | 66 | II-III | +++ | +++ | +++ | ++ | ++ | ++ | neointima |
| M | 49 | V | +++ | +++ | +++ | ++ | + | + | shoulder, activated |
| | | | | | | | | | media, neointima |
| F | 49 | VI | ++ | ++ | ++ | ++ | ++ | ++ | shoulder, activated |
| | | | | | | | | | media, neointima |
| M | 66 | VI | ++ | ++ | ++ | ++ | ++ | ++ | shoulder, activated |
| | | | | | | | | | media, neointima |

M: male; F: female; yrs: years; AHA: American Heart Association Classification; +: low expression, ++: moderate expression, +++: high expression;
^{†}shown in FIG. 20;
^{‡}shown in FIG. 21.

### Example 6

### Use of C-DIM-Eluting Cuffs to Prevent Atherosclerotic Lesion (Containing Smooth Muscle Cells and Inflammatory Cells) Formation in Mice

TR3, MINOR and NOT are expressed in human atherosclerotic lesions in smooth muscle cells, endothelial cells and also in a subset of macrophages. Moreover, the expression of TR3, MINOR and NOT is strongly enhanced upon activation of cultured macrophages, both in primary human macrophages and in the monocytic/macrophage cell line THP-1. We have shown that TR3-like factors inhibit cytokine release and reduce lipid loading of activated macrophages and consequently may delimit the formation of atherosclerotic lesions.

To analyse the effect of C-DIM-compounds on the formation of macrophage-containing atherosclerotic lesions a similar experiment was performed as described in Example 3 and 4, except that ApoE^{-/-} or ApoE*3Leiden mice are applied. ApoE^{-/-} or ApoE*3Leiden mice are exposed to a cholesterol-rich diet and subsequently a perivascular cuff is placed around the femoral artery, which results in accelerated atherosclerosis. Within 2-4 weeks a macrophage- and smooth muscle cell-rich lesion is formed within the cuffed artery, as described by Lardenoye J. H. et al Circ. Res. (2000) 87:248-53.

### Materials and Methods

### Animals

In these experiments ApoE^{-/-} mice or ApoE*3Leiden mice are applied. Eight to 12 week old mice were placed 4 weeks prior to surgery on a cholesterol-enriched high-fat diet to improve intestinal cholesterol uptake and suppress bile acid synthesis.

### Drug-Eluting Cuffs

C-DIM eluting cuffs are made by mixing DIM at 70° C. with polycaprolactene and casting a tubing (0.5 mm inner diameter, 1.0 mm outer diameter). Described in detail in Pires et al., Biomaterials. 2005; 26:5386-94.

### Femoral Artery Cuff Placement

Mice are anaesthetized with an intraperitoneal injection with a solution of Midazolam (12.5 mg/kg bodyweight) and Hypnorm (0.01 ml/mouse). The left-femoral artery is isolated from surrounding tissue, loosely sheathed with a 2.0-mm cuff made of polycaprolactene and polyethylene glycol 0.5 mm inner diameter, 1.0 mm outer diameter was placed loosely around the femoral artery and tied in place with a 6-0 suture. The cuff is wider than the vessel and does not obstruct blood flow.

The right femoral artery is dissected from surrounding tissue (sham-operated), but a cuff is not placed. The femoral arteries are replaced, and the wounds are sutured. After recovery from anaesthesia, the animals are given the cholesterol-enriched high-fat diet and water ad libitum. The mice are either treated with bare, control cuffs or with C-DIM-eluting cuffs, in each group 6 mice are included. Described in detail in Pires et al., Biomaterials. 2005; 26:5386-94.

### Histological Assessment of Intimal Lesions

After 2 to 4 weeks mice are anaesthetized, the thorax is opened and mild pressure-perfusion (100 mmHg) with 3.7% formaldehyde in 0.9% NaCl (wt/vol) for 10 min is performed by cardiac punctures. After perfusion, the femoral artery is harvested, fixed overnight and paraffin-embedded. Serial sections (5 mm thick) are used throughout the entire length of the cuffed femoral artery for histological analysis.

### Morphological Quantification in Sections of Cuffed Femoral Artery

Paraffin sections are stained with haematoxilin/eosin and ten equally spaced (200 mm distance) cross sections are used to quantify intimal lesion. Using image analysis software (Leica, Qwin) total cross-sectional medial areas are measured between the external and internal elastic lamina; total cross-sectional intimal area is also measured between the endothelial cell monolayer and the internal elastic lamina. smooth muscle cells and macrophages are visualized with cell-type specific antibodies; the monoclonal antibody 1A4 (Dako, Glastrup, Denmark) detecting SM alpha-actin and Mac-3 (Accurate Chemicals) to detect monocytes and macrophages, respectively

### Statistical Analysis

Statistical analyses are performed with SPSS, version 10.0.5 software. Experimental values are expressed as mean SEM. The significance of differences is determined by using the nonparametric Mann-Whitney 2-tailed U test and expressed as a probability value.

### Results

It is found that C-DIM-derivatives when applied from a drug-eluting cuff inhibit the formation of atherosclerotic lesions statistically significant. Both the contribution of macrophages and of smooth muscle cells in the lesions is reduced in the C-DIM-eluting cuffs compared to the bare cuffs. These data support potential application of DIM in drug-eluting intravascular stents in humans.

1-20. (canceled)

21. A method of making a medicament for the treatment of atherosclerosis and atherosclerosis-related cardiovascular disease, comprising combining at least one pharmaceutically acceptable excipient with at least one agonist of the nuclear receptors selected from the group consisting of TR3, MINOR and NOT.

22. The method as claimed in claim 21, wherein the atherosclerosis-related cardiovascular disease is in-stent restenosis, vein-graft disease, transplantation arteriosclerosis and/or arteriovenous shunt failure.

23. The method as claimed in claim 21, wherein the agonist is a compound of the formula: 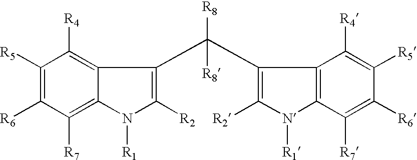
wherein:
R₁, R₂, R₄, R₅, R₆, R₁′, R₂′, R₄′, R₅′, R₆′, and R₇′ are each independently selected from the group consisting of hydrogen, a halogen, a linear C₁-C₁₀ alkyl group, a branched C₁-C₁₀ alkyl group, an alkoxy group containing one to ten carbon atoms, and a nitro group; and
R₈ and R₈′ are each independently selected from the group consisting of hydrogen, a linear C₁-C₁₀ alkyl group, a branched C₁-C₁₀ alkyl group, a cycloalkyl group containing one to ten carbon atoms, and an aryl group.

24. The method as claimed in claim 23, wherein R₁, R₂, R₄, R₅, R₆, R₁′, R₂′, R₄′, R₅′, R₆′, and R₇′ are each hydrogen, and at least one of R₈ and R₈′ is a branched alkyl group, a cycloalkyl group or an aryl group.

25. The method as claimed in claim 23, wherein R₈ and R₈′ are each individually hydrogen, methyl, C₆H₅, C₆H₄OH, C₆H₄CH₃, C₆H₄CF₃, C₁₀H₇, C₆H₄C₆H₅ or C₆H₄OCH₃.

26. The method as claimed in claim 25, wherein one of R₈ and R₈′ is hydrogen and the other is C₆H₅, C₆H₄CF₃, or C₆H₄OCH₃.

27. The method as claimed in claim 21, wherein the treatment is effected by means of a stent that has the medicament incorporated therein and/or coated thereon.

28. The method as claimed in claim 21, wherein treatment is effected by means of a vascular coating.

29. The method as claimed in claim 28, wherein the vascular coating is in the form of a cuff for the graft vein.

30. The method as claimed in claim 28, wherein the vascular coating is in the form of a liquid coating that is applied to the graft vein fixated thereon prior to implantation.

31. The method as claimed in claim 28, wherein the coating is pluronic gel comprising the medicament.

32. A medical device capable of eluting an agonist of one or more of the nuclear receptors TR3, MINOR and NOT for use in the treatment of atherosclerosis and/or atherosclerosis-related cardiovascular disease, comprising a medical device having at least one agonist of one or more of the nuclear receptors TR3, MINOR and NOT incorporated therein.

33. The medical device as claimed in claim 32, wherein the atherosclerosis-related cardiovascular disease is in-stent restenosis, vein-graft disease, transplantation arteriosclerosis and/or arteriovenous shunt failure.

34. The medical device as claimed in claim 33, wherein the agonist is a compound of the formula: 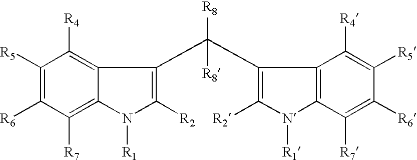
wherein:
R₁, R₂, R₄, R₅, R₆, R₁′, R₂′, R₄′, R₅′, R₆′, and R₇′ are each independently selected from the group consisting of hydrogen, a halogen, a linear C₁-C₁₀ alkyl group, a branched C₁-C₁₀ alkyl group, an alkoxy group containing one to ten carbon atoms, and a nitro group; and
R₈ and R₈′ are each independently selected from the group consisting of hydrogen, a linear C₁-C₁₀ alkyl group, a branched C₁-C₁₀ alkyl group, a cycloalkyl group containing one to ten carbon atoms, and an aryl group.

35. The medical device as claimed in claim 34, wherein R₁, R₂, R₄, R₅, R₆, R₁′, R₂′, R₄′, R₅′, R₆′, and R₇′ are each hydrogen, and at least one of R₈ and R₈′ is a branched alkyl group, a cycloalkyl group or an aryl group, combining at least one pharmaceutically acceptable excipient with one or more of the nuclear receptors selected from the group consisting of TR3, MINOR and NOT.

36. The medical device as claimed in claim 34, wherein R₈ and R₈′ are each individually hydrogen, methyl, C₆H₅, C₆H₄OH, C₆H₄CH₃, C₆H₄CF₃, C₁₀H₇, C₆H₅ or C₆H₄OCH₃.

37. The medical device as claimed in claim 36, wherein one of R₈ and R₈′ is hydrogen and the other is C₆H₅, C₆H₄CF₃, or C₆H₄OCH₃.

38. The medical device as claimed in claim 32, wherein the agonist is incorporated in and/or coated on the medical device.

39. The medical device as claimed in claim 32, wherein the medical device is a stent.

40. The medical device as claimed in claim 32, wherein the medical device is a cuff.
